(19) United States
(12) Patent Application Publication
(10) Pub. No.: US 2023/0241046 A1
(43) Pub. Date: Aug. 3, 2023

(54) SOLID DISPERSION OF OPICAPONE

(71) Applicant: BIAL - PORTELA & CA, S.A., S. Mamede do Coronado (PT)

(72) Inventors: Teófilo Cardoso De Vasconcelos, S. Mamede do Coronado (PT); Pedro Miguel Costa Barrocas, S. Mamede do Coronado (PT); João Filipe Dias Teixeira, S. Mamede do Coronado (PT)

(21) Appl. No.: 18/013,303

(22) PCT Filed: Jul. 28, 2021
(86) PCT No.: PCT/PT2021/050025

§ 371 Date: Dec. 28, 2022

(30) Foreign Application Priority Data
Jul. 28, 2020 (GB) 2011709.9

(51) Int. Cl. A61K 31/4439; A61K 47/32; A61K 47/36

(52) U.S. Cl. CPC A61K 31/4439; A61K 47/32; A61K 47/36

(57) ABSTRACT

This invention relates to kinetically soluble and bioavailable forms of opicapone which exhibit good chemical and solid state stability. In particular, the invention relates to a solid dispersion comprising amorphous opicapone and one or more polymers, wherein the weight ratio of the amorphous opicapone to the one or more polymers ranges from 1:1 to 1:5.

### FIELD OF THE INVENTION

This invention relates to the formulation of opicapone as a medicinal product. In particular, the invention relates to a solid dispersion comprising amorphous opicapone and one or more polymers, wherein the weight ratio of the amorphous opicapone to the one or more polymers ranges from 1:1 to 1:5.

### BACKGROUND OF THE INVENTION

Levodopa (L-DOPA) has been used in clinical practice for several decades in the symptomatic treatment of various conditions, including Parkinson's disease. L-DOPA is able to cross the blood-brain barrier, where it is then converted to dopamine by the enzyme amino acid decarboxylase (AADC), thus increasing dopamine levels in the brain. However, conversion of L-DOPA to dopamine may also occur in peripheral tissues, possibly causing adverse effects. Therefore, it has become standard clinical practice to co-administer a peripheral AADC inhibitor, such as carbidopa or benserazide, which prevents conversion to dopamine in peripheral tissues. It is also known that inhibitors of the enzyme catechol-O-methyltransferase (COMT) may provide clinical improvements in patients afflicted with Parkinson's disease undergoing treatment with L-DOPA, since COMT catalyses the degradation of L-DOPA to the inactive metabolite 3-O-methyldopa.

Opicapone is a potent and long-acting COMT inhibitor. It is bioactive, bioavailable and exhibits low toxicity. Thus, opicapone has potentially valuable pharmaceutical properties in the treatment of some central and peripheral nervous system disorders where inhibition of COMT may be of therapeutic benefit, such as, for example, mood disorders; movement disorders, such as Parkinson's disease, parkinsonian disorders and restless legs syndrome; gastrointestinal disturbances; oedema formation states; and hypertension. The development of opicapone is described in L. E. Kiss et al, J. Med. Chem., 2010, 53, 3396-3411 and it was approved, in combination with L-DOPA, for the treatment of Parkinson's disease in the EU in June 2016 and the US in April 2020.

Further research has focused on optimising opicapone into a stable and bioavailable form. For example, WO 2009/116882 describes various polymorphs of opicapone, with polymorph A being both kinetically and thermodynamically stable. WO 2010/114404 and WO 2010/114405 describe stable opicapone formulations used in clinical trials. In particular, WO 2010/114405 identified excipients in which opicapone displayed enhanced chemical stability compared to phosphate and polyvinylpyrolidone (PVP) derivatives used in early phase clinical trials. WO 2013/089573 describes optimised methods for producing opicapone using simple starting materials and with good yields.

Methods of improving bioavailability of active pharmaceutical ingredients (APIs), especially less water-soluble synthetic molecules are known. The article “Techniques of Bioavailability Enhancement of BCS Class II Drugs: A Review” (Singh et al, Int. J. Pharm. Sci. Res., 2013, 2, 1092-1101) considers over 30 possible methods to improve bioavailability. These are broadly categorised as “traditional techniques” such as use of co-solvents, micronisation, amorphous forms, surfactants and use of functional polymers; “newer and novel techniques” such as nanoparticle technology, lipid-based delivery systems; and “solid dispersion systems” such as simple eutectic mixtures, various solid solutions and glass solutions/suspensions. The review “Technologies to Improve the Solubility, Dissolution and Bioavailability of Poorly Soluble Drugs” (Kanikkannan, J. Anal. Pharm. Res., 2018, 7, 00198) clusters technologies into four groups: “particle size reduction” such as micronisation and nanosuspension; “solid dispersions” formed by spray drying or hot melt extrusion; “lipid based delivery systems”; and “inclusion complexes”. This review discusses both the potential advantages and negative effects of each technique. For example, particle size reduction can be simple and increase dissolution, but is not suitable for all APIs and can chemically destabilise the API. Likewise, solid dispersion can be produced with a wide range of excipients, but can cause chemical instability of the API and requires solvents. Therefore, the net benefit of any specific technology with respect to a specific API is unpredictable.

WO 2009/108077 discloses solid dosage forms for release of poorly water-soluble APIs of Biopharmaceutics Classification System (BCS) class II, wherein the solid dosage form comprises the API, an amorphous carrier and a surfactant. Opicapone is disclosed as one possible API, but no specific opicapone formulation is disclosed.

In addition to describing optimised methods for producing opicapone, WO 2013/089573 also discloses that when recrystallised opicapone is ball milled or micronized through spiral jet mills, opicapone with the desired size for good oral bioavailability can be obtained. This effect is supported by the poster abstract “Relative Bioavailability of Opicapone from Two Different Formulations in Healthy Subjects: The In Vivo Effect of Particle Size” (R. Lima et al, AAPS Annual Meeting, Orlando, 2015), which describes a phase I clinical trial in healthy volunteers comparing the bioavailability (AUC_{0-inf} and Cₘₐₓ) of micronised and non-micronised crystalline opicapone. Therefore, the preferred opicapone form for clinical use is based on a pharmaceutical product as described in WO 2013/089573.

However, there remains a need for a pharmaceutical formulation comprising opicapone which exhibits improved kinetic solubility as well as improved oral bioavailability and/or improved pharmacokinetic parameters (e.g. AUC and Cₘₐₓ) whilst retaining good chemical stability of the API opicapone.

### SUMMARY OF THE INVENTION

The present inventors have now identified particular formulations comprising opicapone with improved kinetic solubility and bioavailability. These formulations comprise amorphous opicapone as a solid dispersion in one or more polymers. The pharmaceutical properties of these solid dispersions can be further improved by including relatively high proportions of the one or more polymers and/or by including surfactants and/or by selecting particular polymers. The solid dispersions have surprisingly improved kinetic solubility and bioavailability compared to known formulations of opicapone and retain surprisingly good chemical stability for this API. For example, the solid dispersions display improved and more consistent kinetic solubility and bioavailability compared to micronized crystalline forms of opicapone. Furthermore, the solid dispersions surprisingly retain chemical stability of the API comparable with the most-stable micronised crystalline formulations of opicapone.

Accordingly, in a first general embodiment, the invention provides a solid dispersion comprising amorphous opicapone and one or more polymers, wherein the weight ratio of the amorphous opicapone to the one or more polymers ranges from 1:1 to 1:5.

In a second general embodiment, the invention provides a pharmaceutical composition comprising a solid dispersion as described above and a pharmaceutically acceptable excipient.

In a third general embodiment, the invention provides a solid dosage form comprising a pharmaceutical composition as described above.

The solid dispersions of the present invention can improve pharmacokinetic parameters (e.g., Cₘₐₓ and/or AUC_{0-24 h}) of opicapone by at least 25% in fasted state simulated intestinal fluid (FaSSIF), and/or by at least 25% in fasted state simulated gastric fluid (FaSSGF), and/or by at least 25% in vivo compared to micronized crystalline opicapone.

The solid dispersions of the present invention can have more consistent pharmacokinetic parameters (e.g., Cₘₐₓ and/or AUC_{0-24 h}) of opicapone in fasted state simulated intestinal fluid (FaSSIF), and/or in fasted state simulated gastric fluid (FaSSGF) and/or in vivo compared to micronized crystalline opicapone.

In a fourth general embodiment, the invention provides a method of modulating opicapone bioavailability by modifying the amounts of PVP:PVA and HPMC polymers in a solid dispersion comprising amorphous opicapone.

### BRIEF DESCRIPTION OF THE DRAWINGS

The invention will now be described in detail with reference to the accompanying drawings, in which:

FIG. 1 shows scanning electron micrographs from scanning electron microscopy (SEM) of opicapone solid dispersion Samples 1 to 4 comprising PVP:PVA (Sample 1), PVP (Sample 2), HPMCAS (Sample 3) and HPMC (Sample 4) with a 200 μm scale bar.

FIG. 2 shows scanning electron micrographs from SEM of opicapone solid dispersion Samples 1 to 4 with a 2 mm scale bar.

FIG. 3 shows X-ray powder diffraction (XRPD) patterns of opicapone solid dispersion Samples 1 to 4.

FIG. 4 shows FaSSGF kinetic solubility traces of opicapone solid dispersion Samples 1 to 4 as well as a micronised crystalline opicapone control.

FIG. 5 shows FaSSIF kinetic solubility traces of opicapone solid dispersion Samples 1 to 4 as well as a micronised crystalline opicapone control.

FIG. 6 shows rat pharmacokinetic data for mean amounts of opicapone in plasma precipitate when dosed with opicapone solid dispersion Samples 1, 4 or 6 comprising PVP:PVA (Sample 1), HPMC (Sample 4), and PVP:PVA and HPMC mixture (Sample 6) or a micronised crystalline opicapone control.

FIG. 7 shows scanning electron micrographs from SEM of opicapone solid dispersion Samples 5 to 9 comprising PVP:PVA only (Sample 5), PVP:PVA and HPMC mixtures (Samples 6 to 8) and HPMC alone (Sample 9) with a 200 μm scale bar.

FIG. 8 shows scanning electron micrographs from SEM of opicapone solid dispersion Samples 5 to 9 with a 2 mm scale bar.

FIG. 9 shows XRPD patterns of opicapone solid dispersion Samples 5 to 9.

FIG. 10 shows FaSSGF kinetic solubility traces of opicapone solid dispersion Samples 5 to 9 as well as a micronised crystalline opicapone control.

FIG. 11 shows FaSSIF kinetic solubility traces of opicapone solid dispersion Samples 5 to 9 as well as a micronised crystalline opicapone control.

FIG. 12 shows kinetic solubility at 4 hours in the FaSSIF kinetic solubility assay for Samples 5 to 9 plotted against the amount of PVP:PVA to HPMC (in percentage PVP:PVA).

FIG. 13 shows scanning electron micrographs from SEM of opicapone solid dispersion Samples 11 to 16 comprising the lauroyl macroglyceride Gelucire 44/14 (Sample 11), Poloxamer 188 (Sample 12), Poloxamer 407 (Sample 13), Vitamin E PEG succinate (Sample 14), sodium lauryl sulphate (Sample 15) and Polysorbate 80 (Sample 16) with a 100 μm scale bar.

FIG. 14 shows scanning electron micrographs from SEM of opicapone solid dispersion Samples 11 to 16 with a 2 mm scale bar.

FIG. 15 shows XRPD patterns of opicapone solid dispersion Sample 10 comprising no surfactant and Samples 11 to 16.

FIG. 16 shows FaSSGF kinetic solubility traces of opicapone solid dispersion Samples 10 to 16 as well as a micronised crystalline opicapone control.

FIG. 17 shows FaSSIF kinetic solubility traces of opicapone solid dispersion Samples 10 to 16 as well as a micronised crystalline opicapone control.

### DETAILED DESCRIPTION OF THE INVENTION

### A. Definitions

The following definitions apply to the terms as used throughout this specification, unless otherwise limited in specific instances.

The term “solid dispersion” refers to a solid product comprising at least two different components, generally a hydrophilic carrier and a hydrophobic API, that are a molecular mixture.

The term “amorphous opicapone” means solid opicapone that is substantially free from crystalline opicapone. Amorphous opicapone preferably contains less than 30%, more preferably less than 20%, even more preferably less than 10% crystalline opicapone. The presence or absence of crystalline forms of opicapone can be assessed by XRPD analysis and/or scanning electron microscopy, as described in section E, below. Preferably, amorphous opicapone exhibits a drop in the XRPD maximum peak intensity of 70%, more preferably 80%, even more preferably 90%, compared to micronised crystalline opicapone. Preferably, the amorphous opicapone exhibits no detectable peaks related to opicapone in the XRPD spectrum between 2 and 40 degrees.

The term “micronised crystalline opicapone” refers to crystalline opicapone polymorph A described in WO 2009/116882 having a particle size distribution (equivalent circular diameter) of D10≥4 μm, D50=10-40 μm and D95≤80 μm.

The term “carrier” refers to components in the solid dispersion that are not API. It includes the one or more polymers and may also include one or more surfactants. It is generally hydrophilic and includes both the polymer components and the surfactant components. Excipients added after the solid dispersion forms are not components of the solid dispersion itself, but may form part of a pharmaceutical composition comprising the solid dispersion.

A “copolymer of N-vinyl-2-pyrrolidone and vinyl acetate” (abbreviated as “PVP:PVA copolymer”) is a water-soluble polymer made from the monomers N-vinylpyrrolidone and vinyl acetate. PVP:PVA copolymers according to the current invention preferably contain at least 5 wt % PVP, more preferably at least 20 wt % PVP, still more preferably at least 40 wt % PVP and most preferably at least 60% PVP. Suitable PVP:PVA copolymers are described in “Biomaterials of PVA and PVP in medical and pharmaceutical applications: Perspectives and challenges” (Teodorescu et al, Biotechnol. Adv., 2019, 37, 109-131). Particularly suitable PVP:PVA copolymers are described in section B, below.

“Hydroxypropylmethylcellulose” (abbreviated as “HPMC”), also known as Hypromellose (INN) is a polymer known to the skilled person. Particularly suitable HPMC polymers are described in section B, below.

“Hydroxypropylmethylcellulose acetyl succinate” (abbreviated as “HPMCAS”) can be produced by the esterification of HPMC with acetic anhydride and succinic anhydride in a reaction medium of a carboxylic acid, such as acetic acid, and using an alkali carboxylate, such as sodium acetate, as catalyst. Particularly suitable HPMCAS polymers are described in section B, below.

“Polyvinylpyrrolidone” (abbreviated as “PVP”), also known as polyvidone or povidone, is a generally water-soluble polymer made from the monomer N-vinylpyrrolidone. Particularly suitable PVP polymers are described in section B, below. Another suitable PVP polymer is crospovidone, also known as polyvinylpolypyrrolidone or polyvinyl polypyrrolidone (abbreviated PVPP).

“Polyethylene glycol-based polymers” include polyethylene glycols (PEGs) (for example PEG 6000), capped PEGs (for example Polyoxyethylene (100) Stearate (Myrj 59P)), and PEG copolymers (for example poly(N-vinyl caprolactam)-poly(vinyl acetate)-poly(ethylene glycol) graft copolymer (Soluplus®). It is noted here that capped PEGs may exhibit surfactant properties (for example in the case of vitamin E polyethylene glycol succinate). In the context of the present invention, a capped PEG (or closely related groups of capped PEGs) with surfactant properties which is present in the solid dispersion in an amount of more than 10 wt % will be considered a polymer. A capped PEG with surfactant properties which is present in the solid dispersion in an amount of 10 wt % or less will be considered a surfactant.

“Acrylate or Methacrylate-based polymers” include polymers of acrylic acid esters, such as poly(methacrylate) (PMA) and poly(ethyl acrylate) (PEA); polymers of methacrylic acid esters, such as poly(methyl methacrylate) (PMMA), poly(ethyl methacrylate) (PEMA) and poly(hydroxyethyl methacrylate) (poly-HEMA); and copolymers of one or more different acrylic and/or methacrylic acid esters, such as Eudragit RLPO which includes ethyl acrylate, methyl methacrylate and quaternary ammonium functionalised ethyl methacrylate as monomers.

Amounts defined in “weight %” (abbreviated as “wt %” or “w/w %”) relate to the percent amount of a particular component in a mixture based on the dry weight of the total mixture.

A “weight ratio” relates to the proportion by weight of two (or more) components in a mixture relative to each other. Unless stated otherwise, a specified weight ratio does not exclude other components from the mixture. For example, a 1:1 weight ratio could relate to two components in a mixture, each present at 40 wt % with 20 wt % of one or more other component.

The terms “fasted state simulated intestinal fluid” (abbreviated as “FaSSIF”) and “fasted state simulated gastric fluid” (abbreviated as “FaSSGF”) refer to bio-relevant dissolution media used to forecast the in vivo performance of APIs, as described in the review “The Use of Biorelevant Dissolution Media to Forecast the In Vivo Performance of a Drug” (Klein, AAPS J., 2010, 12, 397-406). The composition of “FaSSGF pH 1.6” is disclosed in Table I of Klein 2010. The composition of “FaSSIF pH 6.5” is disclosed in Table II of Klein 2010.

The term “pharmacokinetic parameters” includes the maximum concentration (Cₘₐₓ) and the area under the concentration curve from time points x to y (AUC_{x-y}). These parameters may be expressed in absolute terms (e.g. mg/mL or mg·h/mL) or relative to a standard control sample (e.g., micronised crystalline opicapone). The pharmacokinetic parameters can be simulated in bio-relevant dissolution media (e.g., FaSSIF or FaSSGF), calculated in relevant animal models (e.g. rats) or calculated in humans.

A “significant increase in bioavailability” of opicapone is defined as an increase in a pharmacokinetic parameter (e.g. AUC_{x-y} and/or Cₘₐₓ) for opicapone, such that the solid dispersion, pharmaceutical composition or solid dosage form exhibiting said increase may no longer be considered bioequivalent to a standard control sample (e.g., a pharmaceutical composition or solid dosage form comprising micronised crystalline opicapone), in particular to a medicinal product approved by the relevant regulatory authorities. The term “bioequivalent” is known to the skilled person and generally refers to a final medicinal product exhibiting pharmacokinetic parameters (e.g. AUC_{x-y} and Cₘₐₓ) in the range of 80 to 125% of standard pharmacokinetic parameters established for the final medicinal product as approved by the relevant regulatory authorities.

The term “more consistent pharmacokinetic parameters” for opicapone means that the variation in one or both of these parameters between multiple runs (e.g. three samples) of a solid dispersion, pharmaceutical composition or solid dosage form produced by the same method is significantly reduced compared to a standard control sample (e.g., a pharmaceutical composition or solid dosage form comprising micronised crystalline opicapone). A significant reduction in variation means a reduction in the standard deviation of one or both of these pharmacokinetic parameters in the solid dispersion, pharmaceutical composition or solid dosage form compared to the same number of batches of a standard control sample (e.g., a pharmaceutical composition or solid dosage form comprising micronised crystalline opicapone). Preferably, the variation is reduced such that pharmacokinetic parameters for all runs of the solid dispersion, pharmaceutical composition or solid dosage form lie within 80.00 to 125.00% of the standard pharmacokinetic parameters.

A “standard control” or “standard control sample” is expected to be the micronised crystalline opicapone currently used in the marketed formulation (or a pharmaceutical composition or solid dosage form thereof). The control will normally contain an equivalent amount of opicapone (e.g. 25 or 50 mg). However, it may be appropriate to include a bioequivalent amount of opicapone, especially when testing the chemical stability of final dosage forms. For example, if the solid dispersion results in a 5-fold increase in bioavailability, it might be more appropriate to compare the chemical stability of a 50 mg solid dosage form of micronised crystalline opicapone with a 10 mg solid dosage form of the solid dispersion.

A “solid dosage form” is a medicinal product providing a defined dosage of opicapone in a solid form suitable to be taken by an individual (e.g. a capsule or tablet). Preferably, the solid dosage form has improved bioavailability or is bioequivalent to a formulation of opicapone which is approved by one or more national regulatory authorities.

Other variations to the disclosed embodiments can be understood and effected by those skilled in the art in practicing the claimed invention, from a study of the disclosure, and the appended claims. In the claims, the word “comprising” does not exclude other elements or steps, and the indefinite article “a” or “an” does not exclude a plurality. The mere fact that certain measures are recited in mutually different dependent claims does not indicate that a combination of these measures cannot be used to advantage.

### B. Solid Dispersions

The invention provides a solid dispersion comprising amorphous opicapone and one or more polymers, wherein the weight ratio of the amorphous opicapone to the one or more polymers ranges from 1:1 to 1:5.

### Advantages of the Invention

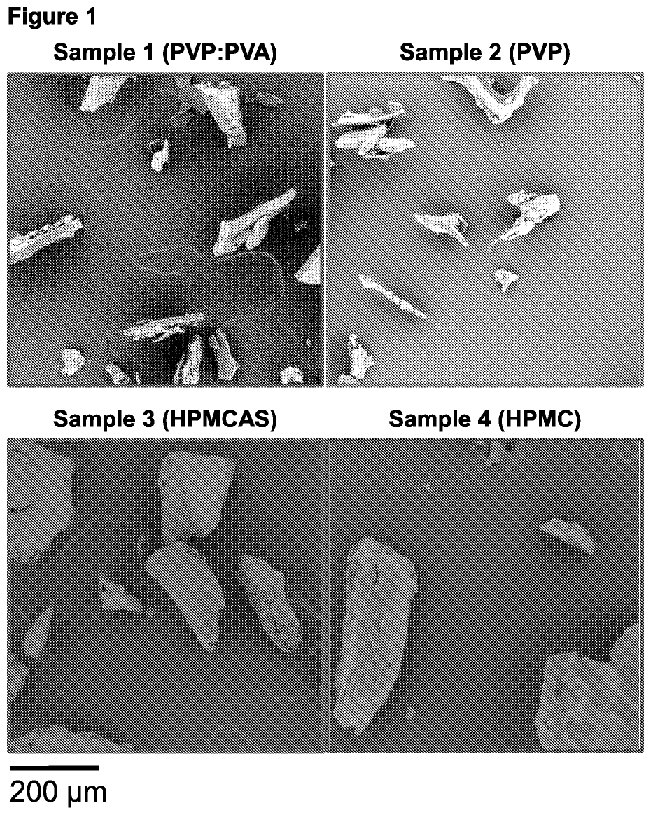

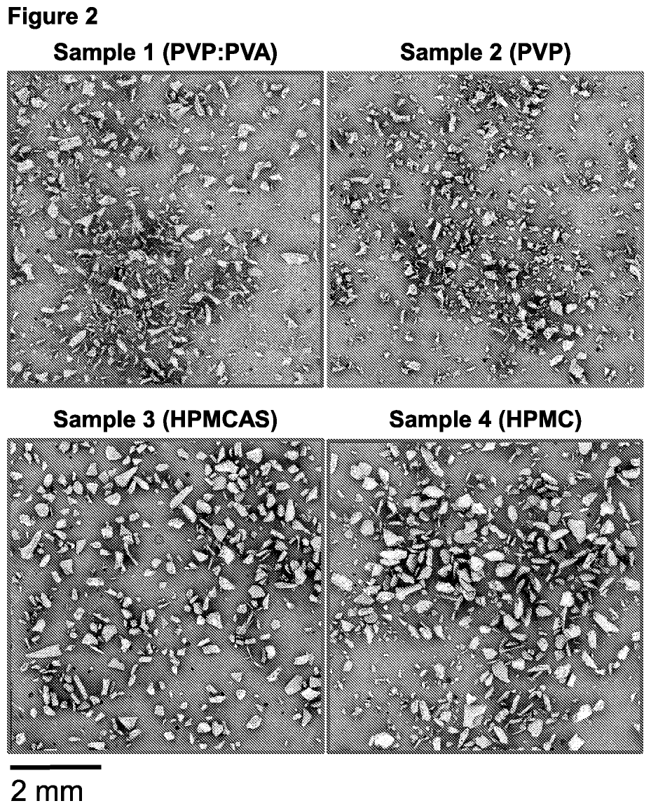

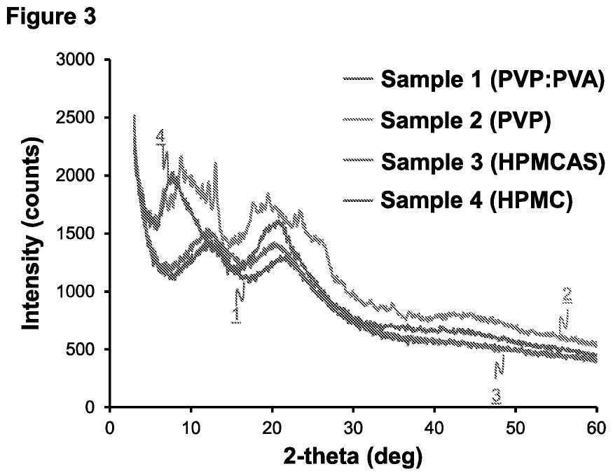

The inventors surprisingly discovered that when opicapone is produced in an amorphous form by being combined with one or more polymers at the specified ratios in the form of a solid dispersion, a significant increase in kinetic solubility and bioavailability was observed compared to a standard control (e.g., micronised crystalline opicapone). This is particularly surprising because micronised crystalline opicapone itself already significantly increases the bioavailability of opicapone compared to the non-micronised crystalline forms. The use of the claimed opicapone solid dispersions allows solid dosage forms to be produced containing lower amounts of opicapone compared to a standard control (e.g., a solid dosage form comprising micronised crystalline opicapone). Solid dispersions (Samples 1 to 4) described in Experiment 1 and including a number of polymers are shown in Table 1 in section F, below, and were characterised by Scanning Electron Microscopy (SEM) at different magnifications (FIG. 1 and FIG. 2) and X-Ray Powder Diffraction (XRPD) analysis (FIG. 3), below.

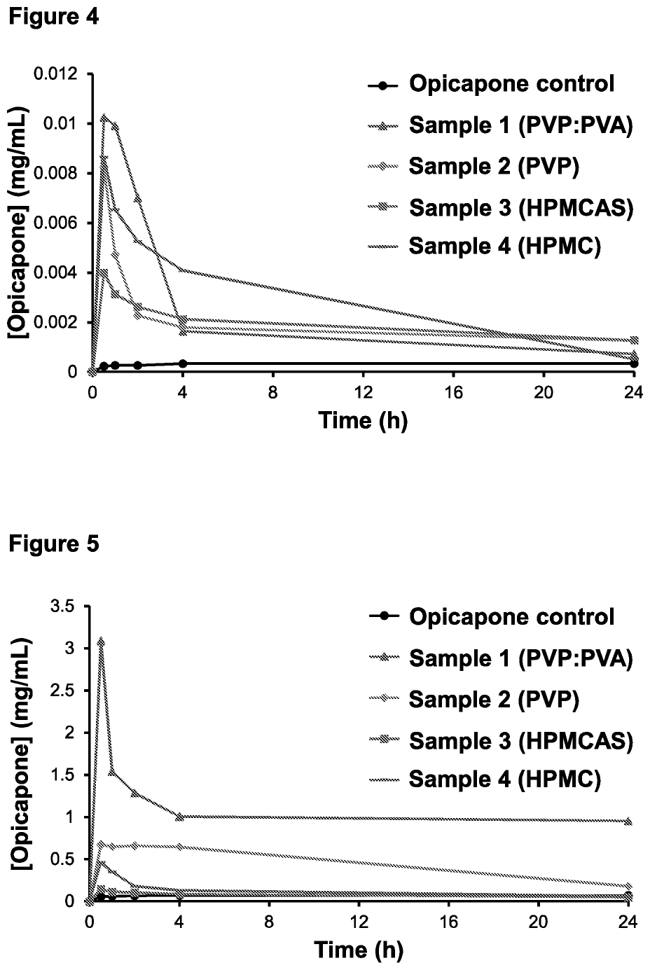

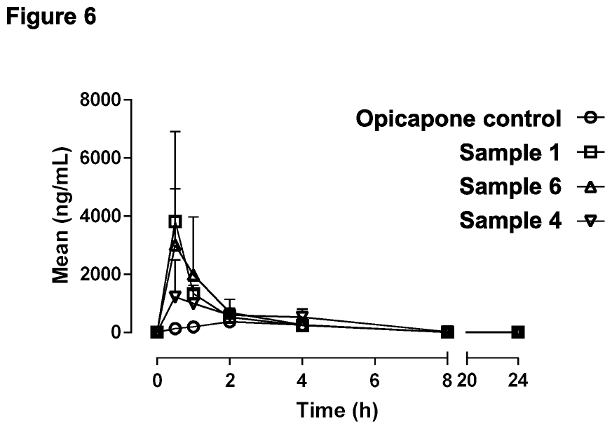

All solid dispersions displayed improved kinetic solubility compared to a standard control (e.g., micronised crystalline opicapone) in a FaSSGF kinetic dissolution assay that mimics dissolution in the stomach (FIG. 4 and Table 2) and FaSSIF kinetic dissolution assay that mimics dissolution in the intestine (FIG. 5 and Table 3). Representative solid dispersions also improved pharmacokinetic parameters in an in vivo rat model compared to a standard control (e.g., micronised crystalline opicapone) (FIG. 6 and Table 4). The improvements in kinetic solubility and bioavailability compared to micronised crystalline opicapone were not related to the size of the particles.

Generally, the Cₘₐₓ and/or AUC_{0-24 h} of opicapone in the amorphous solid dispersions is/are increased by at least 25% in FaSSIF compared to micronized crystalline opicapone. Alternatively, or additionally, the Cₘₐₓ and/or AUC_{0-24 h} of opicapone in the amorphous solid dispersions is/are increased by at least 25% in FaSSGF compared to micronized crystalline opicapone. Additionally or alternatively, the Cₘₐₓ and/or AUC_{0-24 h} of opicapone in the amorphous solid dispersions is/are increased by at least 25% in vivo, preferably in rats or humans, most preferably in humans. In many cases, one or more of these values will be increase by 50%, 100%, 200% or even 500% (with larger values being increasingly preferred).

Additionally, the inventors found that the solid dispersion maintained good chemical stability compared to a standard control (e.g., micronised crystalline opicapone). The chemical stability is shown in Table 8, where only 0.1 to 0.5% of opicapone-related impurities were identified by HPLC after storage for 6 to 12 months at room temperature (25° C.) and 60% RH. This is surprising because crystalline APIs are generally considered to be more stable than APIs in an amorphous form. In particular, the purity of opicapone after storage for 6 to 12 months at room temperature (25° C.) and 60% RH remained at 99.5 to 99.9% in the solid dispersions, suggesting they could be suitable to use in marketed pharmaceutical formulations. Therefore, in a preferred embodiment, the opicapone is chemically stable for 6 months, preferably 12 months, at room temperature (25° C.) and 60% RH. Preferably, the opicapone is 99% pure or greater after storage under these conditions, more preferably 99.5% pure or greater after storage under these conditions.

Given that the improved kinetic dissolution and bioavailability correlates with the amorphous nature of the opicapone, it is important the API remains amorphous during long-term storage. Preferably, the amorphous opicapone remain amorphous for 6 months, preferably 12 months, at room temperature (25° C.) and 60% RH. The definition of “amorphous opicapone” is given above in Section A. More preferably, opicapone is both chemically stable and remains amorphous under these conditions.

### Weight Ratio

The weight ratio of the amorphous opicapone to the one or more polymers in the solid dispersion ranges from 1:1 to 1:5, more preferably 1:2 to 1:5 and even more preferably 1:3 to 1:5. Most preferably, the weight ratio of the amorphous opicapone to the one or more polymers in the solid dispersion is about 1:4. These ratios provide excellent bioavailability compared to a standard control (e.g., micronised crystalline opicapone) and good chemical stability compared to equivalent solid dispersions with higher relative amounts of amorphous opicapone. Furthermore, these ratios are predicted to remain amorphous over longer periods compared to equivalent solid dispersions with higher relative amounts of opicapone.

### Polymer Selection

In another preferred embodiment that can be combined with different weight ratios of opicapone to the one or more polymers, the one or more polymers are selected from the group consisting of: a copolymer of N-vinyl-2-pyrrolidone and vinyl acetate (PVP:PVA), hydroxypropylmethylcellulose (HPMC), hydroxypropylmethylcellulose acetyl succinate (HPMCAS), an acrylate- or methacrylate-based polymer, a polyethylene glycol-based polymer and polyvinylpyrrolidone (PVP).

FIG. 4 and FIG. 5 confirm that representative examples of these particular polymers provided improved kinetic solubility for amorphous opicapone compared to a standard control of micronised crystalline opicapone in both the FaSSGF and FaSSIF assays. Table 2 and Table 3 confirm similar effects in predicted bioavailability. In particular, FIG. 4 and Table 2 confirm that HPMC gives the largest improvement in kinetic solubility in FaSSGF. This might facilitate passing of opicapone to the intestine, where it is primarily absorbed. FIG. 5 and Table 3 confirm that PVP and PVP:PVA, especially PVP:PVA, give the largest improvement in kinetic solubility in FaSSIF. The levels of kinetic solubility are generally higher in the FaSSIF assay and opicapone is generally believed to dissolve and absorb in the intestine. Therefore, rapid dissolution in FaSSIF is generally considered most important. The inventors saw good correlation between the predicted bioavailability in the FaSSIF and the bioavailability in the in vivo rat model.

Therefore, in a particularly preferred aspect of this embodiment, the one or more polymers are selected from the group consisting of: PVP, PVP:PVA and HPMC. For example, the polymer may be PVP. For example, the polymer may be PVP:PVA. For example, the polymer may be HPMC.

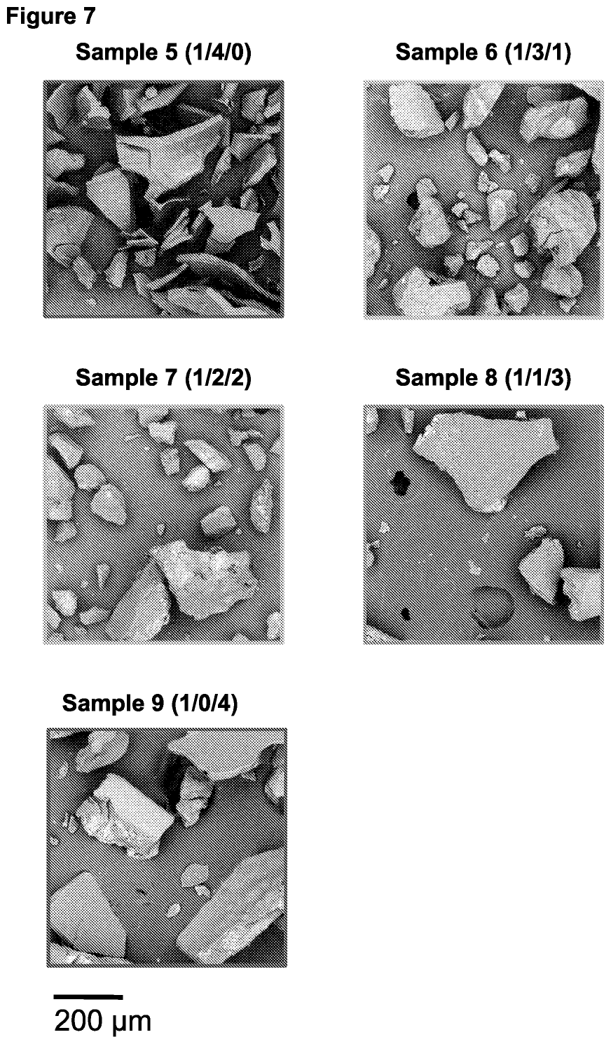

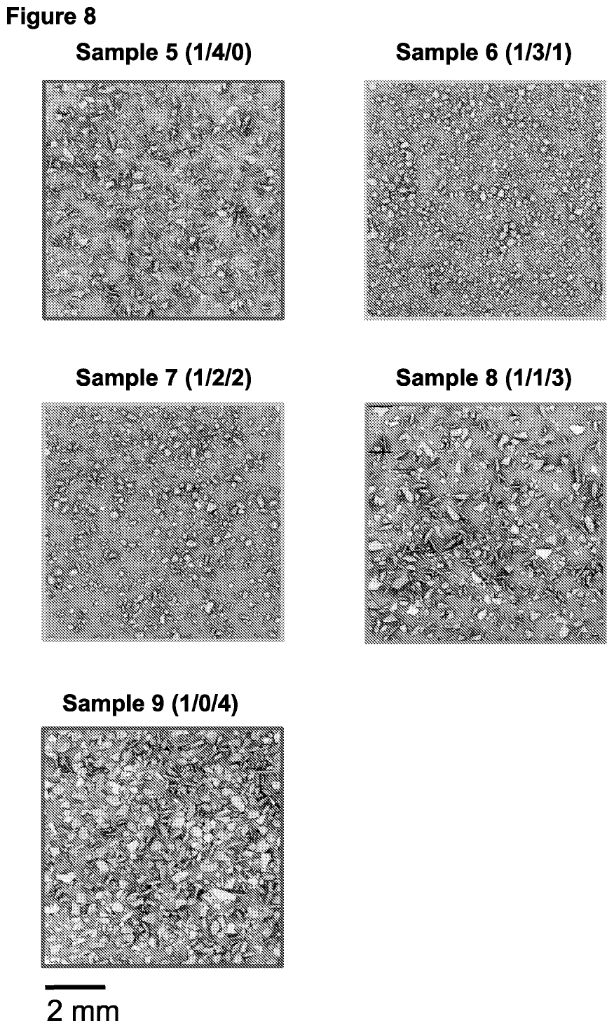

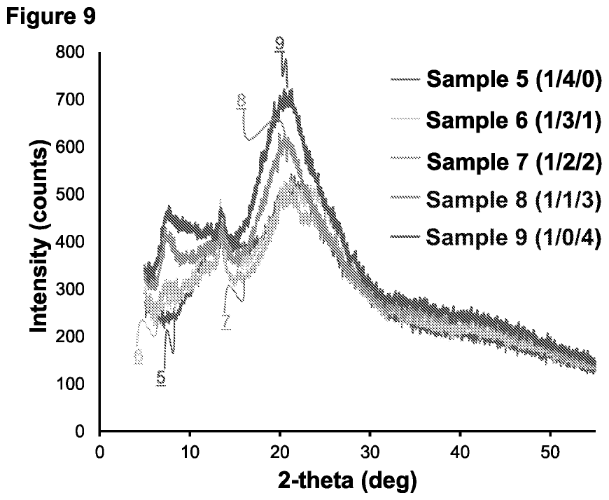

Based on the discovery that (a) solid dispersions of amorphous opicapone had improved kinetic solubility and pharmacokinetic parameters compared to a standard control of micronised crystalline opicapone, and (b) that the level of improvement varied, the inventors performed further experiments to identify combinations of polymers that resulted in particularly large increases in kinetic solubility and bioavailability. The inventors prepared solid dispersions comprising amorphous opicapone and different polymer mixtures. Those comprising a mixture of PVP:PVA and HPMC were found to have excellent kinetic solubility and pharmacokinetic parameters as well as good chemical stability. Solid dispersions (Samples 5 to 9) described in Example 2 and including different ratios of PVP:PVA and HPMC are shown in Table 5 in section F, below, and were characterised by scanning electron microscopy (FIG. 7 and FIG. 8) and X-ray powder diffraction analysis (FIG. 9), below.

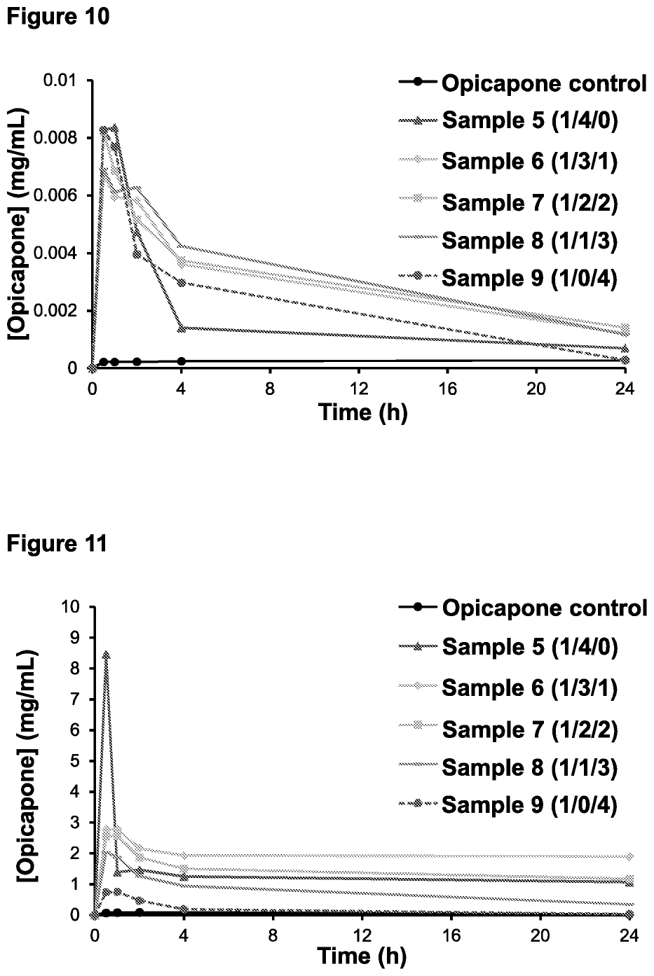

All solid dispersions displayed improved kinetic solubility compared to a standard control (e.g., micronised crystalline opicapone) in a FaSSGF (FIG. 10) and FaSSIF dissolution assay (FIG. 11).

All mixtures (Sample 6, Sample 7 and Sample 8) displayed improved FaSSGF dissolution compared to the PVP:PVA sample (Sample 5) and HPMC sample (Sample 9). Table 6 confirms a similar effect in predicted bioavailability.

Therefore, in another particularly preferred aspect of this embodiment, the one or more polymers are a mixture of PVP:PVA and HPMC. Whilst this combination of polymers can be included alongside other polymers, in this embodiment, it is preferred that PVP:PVA and/or HPMC make up at least 80%, more preferably at least 90%, even more preferably at least 95% of the total polymer. In a most preferred example of this embodiment, the one or more polymers consist entirely of a mixture of PVP:PVA and HPMC.

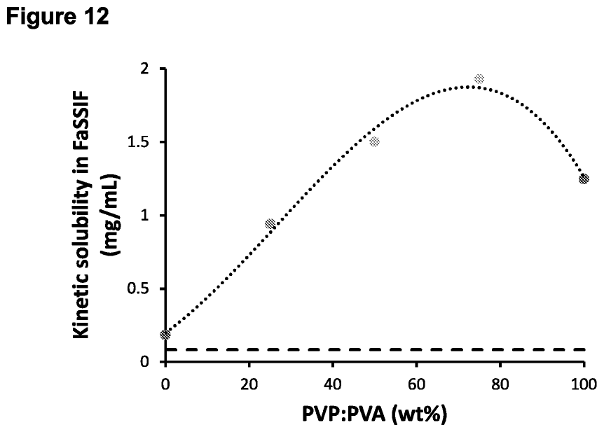

The inventors further discovered that varying the ratio of PVP:PVA and HPMC resulted in improvement in FaSSIF kinetic solubility displaying a parabolic trend with an apex around the 3:1 PVP:PVA to HPMC ratio (i.e. 75% PVP:PVA). This effect is shown in FIG. 12, based on the 4-hour time point, with the x-axis express at amount of PVP:PVA relative to the total polymer (%). Table 7 confirms a similar effect in predicted bioavailability.

Therefore, in an even more preferred aspect of this embodiment, when mixtures of PVP:PVA and HPMC are used, the weight ratio of PVP:PVA to HPMC ranges from 1:4 to 20:1, more preferably from 1:1 to 10:1, even more preferably from 2:1 to 4:1. Most preferably, the weight ratio of PVP:PVA to HPMC is about 3:1.

The identification of a non-linear trend between the weight ratio of PVP:PVA to HPMC and the opicapone bioavailability allows the bioavailability to be modulated by varying the weight ratio of PVP:PVA. Generally, this will be used to maximise the bioavailability, but could be used, for example, to ensure bioequivalence with a particular formulation or to ensure lower doses in patients with lower body weight without substantially altering the components of the solid dispersion.

Therefore, another preferred embodiment relates to a method of modulating opicapone bioavailability by modifying the amounts of PVP:PVA and HPMC polymers in a solid dispersion comprising amorphous opicapone. Preferably, mixtures of PVP:PVA and HPMC are used. When mixtures of PVP:PVA and HPMC are used, the weight ratio of PVP:PVA to HPMC preferably ranges from 1:4 to 20:1, more preferably from 1:1 to 10:1, even more preferably from 2:1 to 4:1. Most preferably, the weight ratio of PVP:PVA to HPMC is about 3:1.

Based on the discovery that different ratios of PVP:PVA to HPMC could alter kinetic solubility and bioavailability, the inventors performed further experiments to identify combinations of polymers that resulted in good kinetic solubility and bioavailability as well as excellent long-term chemical stability. Furthermore, the inventors assess whether opicapone remained amorphous in these combinations of polymers.

Provisional experiments performed on Samples 5, 6 and 9 at 70° C. and 75% RH for 19 days confirmed all samples maintained good chemical stability with opicapone chemical purity decreasing from 99.5-99.9% at day 0 to 95.7-96.6% by day 19. This confirmed that the opicapone in all solid dispersions remained chemically stable in spite of their different kinetic solubility.

Long-term stability testing was performed at room relative humidity (RH) of 60% RH and temperature (25° C.) for 12 months in amber glass bottles. Samples 5, 6 and 9 were tested by visual inspection, XRPD and assayed for purity by HPLC. Such tests are more relevant for the actual chemical stability of the API, opicapone, as well as for assessing its amorphous character.

Solid dispersions (Samples 5, 6 and 9) described in Example 2 and including PVP:PVA alone (Sample 5), a 3/1 ratio of PVP:PVA and HPMC (Sample 6) and HPMC alone (Sample 9) are shown in Table 5 and their stability is shown in Table 8 in section F, below. All samples remained yellow solids and displayed minimal total impurities after 12 months.

As mentioned above, the chemical stability is shown in Table 8, where only 0.1 to 0.5% of opicapone-related impurities were identified by HPLC after storage for 6 to 12 months at room temperature (25° C.) and 60% RH. The purity of opicapone after storage for 6 to 12 months at room temperature (25° C.) and 60% RH remained at 99.5 to 99.9% in the solid dispersions. Therefore, in a preferred embodiment, the opicapone is chemically stable for 6 months, preferably 12 months, at room temperature (25° C.) and 60% RH. Preferably, the opicapone is 99% pure or greater after storage under these conditions, more preferably 99.5% pure or greater after storage under these conditions.

The solid state stability of the amorphous form of opicapone (i.e., the maintenance of opicapone in a fully amorphous form) is also shown in Table 8. The long-term stability data confirm that only the sample containing PVP:PVA (Sample 5) remained fully “amorphous opicapone” under these conditions, meaning it is substantially free from crystalline opicapone. Most preferably, the amorphous opicapone remain amorphous for 6 months, preferably 12 months, at room temperature (25° C.) and 60% RH.

Therefore, in a most preferred aspect of this embodiment, where good bioavailability, excellent long-term chemical stability and excellent long-term retention of amorphous character are required, the one or more polymers is PVP:PVA.

### Surfactants

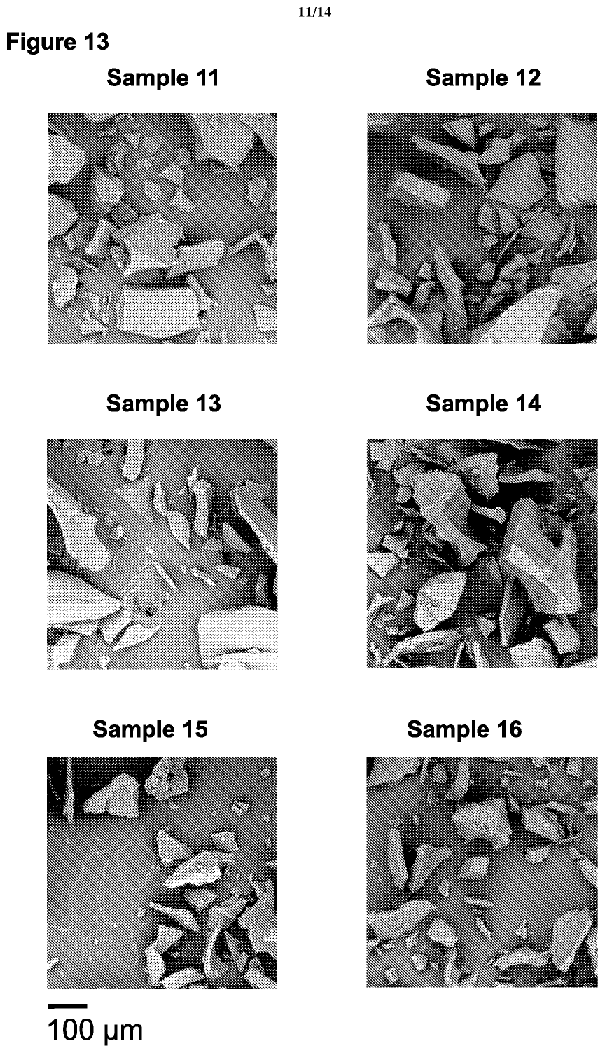

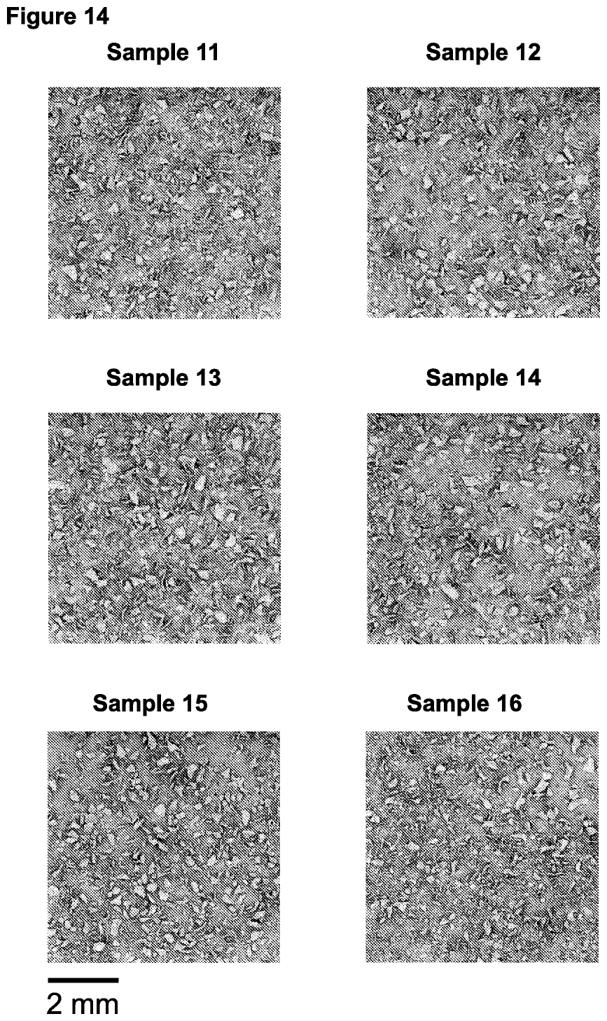

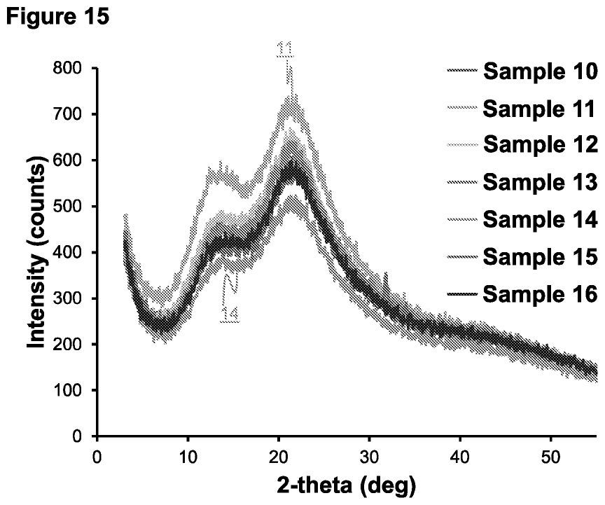

The inventors then assessed the effect of surfactants on the kinetic solubility of amorphous solid dispersions of opicapone using the FaSSGF and FaSSIF kinetic dissolution assays. Solid dispersions (Samples 10 to 16) described in Example 3 and including different surfactants are shown in Table 9 in section F, below, and were characterised by scanning electron microscopy (FIG. 13 and FIG. 14) and X-ray powder diffraction analysis (FIG. 15), below.

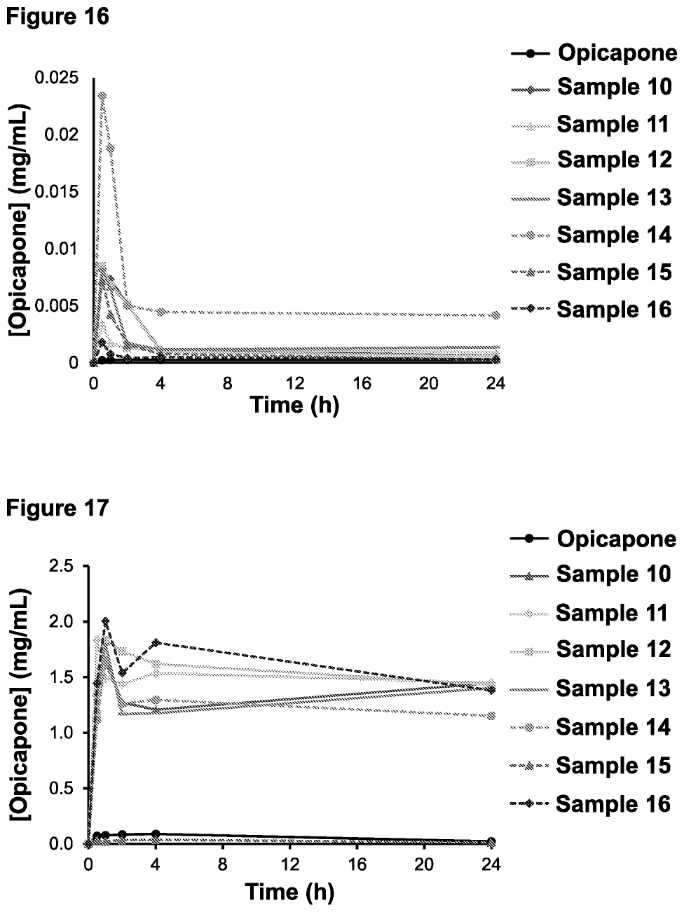

FIG. 16 and FIG. 17 confirm surfactants can provide improved kinetic solubility for amorphous opicapone compared to a standard control of micronised crystalline opicapone in both the FaSSGF and FaSSIF assays, respectively. Table 10 and Table 11 confirm similar effects in predicted bioavailability. All solid dispersions displayed improved kinetic solubility compared to a standard control (e.g., micronised crystalline opicapone) in a FaSSGF kinetic dissolution assay that mimics dissolution in the stomach (FIG. 16 and Table 10).

Therefore, in another preferred embodiment that can be combined with different weight ratios of the amorphous opicapone to the one or more polymers and with different specific polymers, the solid dispersion comprises a surfactant. In particular, the surfactant is selected from the group consisting of lauroyl macrogolglycerides, poloxamers, polysorbates, organosulphates and vitamin E polyethylene glycol succinate.

In the FaSSIF kinetic dissolution assay, which mimics dissolution in the intestine (FIG. 17 and Table 11), most surfactants substantially improved kinetic solubility (Samples 11 to 14 and Sample 16) with the exception of sodium lauryl sulphate (SLS) (Sample 15).

The solid dispersions comprising surfactants generally enhance kinetic solubility in FaSSIF over 20-fold, with the exception of SLS.

A distinguishing feature between SLS and the other surfactants tested is that SLS is negatively charged in solution whereas all other surfactants were non-ionic.

Therefore, in a more preferred aspect of this embodiment, the surfactant is non-ionic. In an alternative aspect, the surfactant is not anionic, in particular the surfactant is not an organosulphate surfactant, specifically, the surfactant is not SLS.

In a more preferred aspect of this embodiment, the surfactant is selected from the group consisting of lauroyl macrogolglycerides, poloxamers, polysorbates, and vitamin E polyethylene glycol succinate, even more preferably from the group consisting of lauroyl macrogolglycerides, poloxamers and polysorbates, and most preferably from the group consisting of lauroyl macrogol-32 glycerides, poloxamer 188 and polysorbate 80.

The amount of surfactant is not particularly limited, but is generally in the range of from 2 to 8 wt % based on the total weight of the solid dispersion, preferably from 3 to 7 wt %, more preferably from 4 to 6 wt %.

The surfactant can be combined with any disclosed polymer, but has been found to be particularly compatible with PVP:PVA copolymers and with mixtures of PVP:PVA and HPMC of any amount.

Based on the discovery that different surfactants could alter kinetic solubility and bioavailability, the inventors performed further experiments to identify combinations of polymers and surfactants that resulted in good kinetic solubility and bioavailability as well as excellent long-term chemical stability and excellent long-term retention of amorphous character.

Provisional experiments performed on Samples 10 to 14 at 70° C. and 75% RH for 19 days confirmed all samples maintained good chemical stability with total impurities increasing from 0.3-0.4% at day 0 to 2.9-5.1% by day 19. This confirmed the opicapone remained 94.9 to 97.1% pure and that all amorphous solid dispersions remained chemically stable in spite of their different kinetic solubility.

Furthermore, long-term stability testing was performed at room relative humidity (RH) of 60% RH and temperature (25° C.) for 6 months in amber glass bottles. Samples 10 to 14 were tested by visual inspection, XRPD and assayed for purity by HPLC. Such tests are more relevant for the actual stability of the active pharmaceutical ingredient, opicapone.

Solid dispersions (Samples 10 to 14) described in Example 3 and including different surfactants are shown in Table 9 and their chemical stability and retention of the amorphous state is shown in Table 12 in section F, below. All samples remained yellow solids, displayed minimal total impurities after 6 months and remained fully “amorphous opicapone” under these conditions, meaning it is substantially free from crystalline opicapone.

Therefore, it is apparent that the presence of surfactant does not reduce stability in spite of improving kinetic solubility. The use of a PVP:PVA copolymer in combination with certain surfactants provides maximum chemical stability, retention of the amorphous state and provides kinetic dissolution comparable with the best mixtures of PVP:PVA and HPMC.

### C. Pharmaceutical Compositions and Solid Dosage Forms Comprising a Solid Dispersion of Amorphous Opicapone

Once it has been established that the solid dispersion is in accordance with the invention, it can be further processed into a final medicinal product. Because the solid dispersions of the invention have improved bioavailability compared to micronised crystalline opicapone, they can be provided in lower dosage form.

Accordingly, the present invention also provides a pharmaceutical composition comprising a solid dispersion as described above and a pharmaceutically acceptable excipient. Such a bulk pharmaceutical composition may then be divided into portions containing a suitable dose of opicapone and further processed to provide a solid dosage from such as a capsule or tablet.

The pharmaceutical composition of the invention can be loaded into capsules. The capsules generally contain 1 to 100 mg of opicapone, preferably 2 to 50 mg of opicapone, more preferably 3 to 20 mg of opicapone.

Examples of capsules according to the present invention with improved bioavailability compared to capsules comprising micronised crystalline opicapone are shown in Table A below:

TABLE A
Contents of capsules with improved bioavailability
compared to micronised crystalline opicapone.

| | Capsule (components in mg) | | | | | | |
|---|---|---|---|---|---|---|---|
| | 1 | 2 | 3 | 4 | 5 | 6 | 7 |
| opicapone | 50 | 50 | 50 | 50 | 50 | 50 | 50 |
| PVP:PVA | 200 | | 150 | 137.5 | 137.5 | 137.5 | 103 |
| PVP | | 200 | | | | | |
| HPMC | | | 50 | | | | 34.5 |
| lauroyl macrogol-32 | | | | 12.5 | | | |
| glycerides | | | | | | | |
| poloxamer 188 | | | | | 12.5 | | |
| polysorbate 80 | | | | | | 12.5 | 12.5 |

Examples of capsules according to the present invention with bioequivalence to 50 mg capsules comprising micronised crystalline opicapone are shown in Table B below:

TABLE B
Contents of capsules with predicted bioequivalence
to 50 mg micronised crystalline opicapone.

| | Capsule (components in mg) | | | | | | |
|---|---|---|---|---|---|---|---|
| | 8 | 9 | 10 | 11 | 12 | 13 | 14 |
| opicapone | 5-20 | 5-20 | 5-20 | 5-20 | 5-20 | 5-20 | 5-20 |
| PVP:PVA | 200 | | 150 | 137.5 | 137.5 | 137.5 | 103 |
| PVP | | 200 | | | | | |
| HPMC | | | 50 | | | | 34.5 |
| lauroyl macrogol-32 | | | | 12.5 | | | |
| glycerides | | | | | | | |
| poloxamer 188 | | | | | 12.5 | | |
| polysorbate 80 | | | | | | 12.5 | 12.5 |

The pharmaceutical composition of the invention can be also be compressed into tablets. The tablets generally contain 1 to 100 mg of opicapone, preferably 2 to 50 mg of opicapone, more preferably 3 to 20 mg of opicapone.

In one exemplary embodiment, the pharmaceutical composition comprises 0.2 to 50 wt % of the solid dispersion (such as those in Table B) and 50 to 99.8 wt % of pharmaceutically acceptable excipient(s), preferably comprising 1 to 15 wt % binder and 33 to 85 wt % filler, and optionally 0.5 to 15 wt % lubricant and/or 1 to 15 wt % disintegrant, such as the following compositions and/or formulations:

| | | |
|---|---|---|
| Solid dispersion (of the present invention) | 0.2-50 | wt % |
| Filler | 35.0-85.0 | wt % |
| Binder | 1.0-15.0 | wt % |
| Lubricant | 1.0-15.0 | wt % |
| Disintegrant | 1.0-15.0 | wt % |
| Solid dispersion (of the present invention) | 30.0-50.0 | wt % |
| Filler | 35.0-60.0 | wt % |
| Binder | 3.0-10.0 | wt % |
| Lubricant | 1.0-10.0 | wt % |
| Disintegrant | 3.0-10.0 | wt % |
| Solid dispersion (of the present invention) | 0.2-35 | wt % |
| Filler | 50.0-85.0 | wt % |
| Binder | 3.0-10.0 | wt % |
| Lubricant | 1.0-10.0 | wt % |
| Disintegrant | 3.0-10.0 | wt % |
| Solid dispersion (of the present invention) | 5-25 | wt % |
| Filler | 60.0-80.0 | wt % |
| Binder | 5.0 -10.0 | wt % |
| Lubricant | 0.5-4.0 | wt % |
| Disintegrant | 4.0-8.0 | wt % |

The final medicament can be administered at regular intervals, for example once daily or once weekly, preferably once daily.

The solid dispersion manufactured according to the method of the invention may be administered alone or in combination with one or more other drugs (for example, a dopamine precursor and/or an AADC inhibitor). Generally, the dopamine precursor and/or AADC inhibitor will be administered as a single formulation in association with one or more pharmaceutically acceptable excipients and will be administered at least 1 hour before or after the pharmaceutical composition manufactured according to the method of the invention.

Pharmaceutical compositions suitable for the delivery of compounds of the present invention and methods for their preparation will be readily apparent to those skilled in the art. Such compositions and methods for their preparation may be found, for example, in “Remington's Pharmaceutical Sciences”, 19th Edition (Mack Publishing Company, 1995). Particularly suitable excipients include fillers, binders, lubricants and/or disintegrants.

Fillers/diluents of the present disclosure include calcium phosphate, dibasic anhydrous (for example, A-TAB™, Di-Cafos A-N™, Emcompress™ Anhydrous, and Fujicalin™); calcium phosphate, dibasic dihydrate (for example, Cafos™, Calipharm™, Calstar™, Di-Cafos™, Emcompress™); and calcium phosphate tribasic (for example, Tri-Cafos™, TRI-CAL™ WG, TRI-TAB™). In a further embodiment, the filler may be chosen from starches, lactose, and cellulose. In at least one embodiment, at least two fillers may be present, for example a combination of starch, lactose, and/or cellulose. A preferred filler is lactose.

Binders of the present disclosure include acacia, alginic acid, carbomer, carboxymethylcellulose sodium, ceratonia, cottonseed oil, dextrin, dextrose, gelatin, guar gum, hydrogenated vegetable oil type I, hydroxyethyl cellulose, hydroxyethylmethyl cellulose, hydroxypropyl cellulose, low substituted hydroxypropyl cellulose, hypromellose, magnesium aluminium silicate, maltodextrin, maltose, methylcellulose, ethylcellulose, microcrystalline cellulose, polydextrose, polyethylene oxide, polymethacrylates, sodium alginate, starch, pregelatinised starch, stearic acid, sucrose and zein. A preferred binder is pregelatinised starch.

Lubricants/flow agents of the present disclosure include calcium stearate, glycerine monostearate, glyceryl behenate, glyceryl palmitostearate, hydrogenated castor oil, hydrogenated vegetable oil type I, magnesium lauryl sulphate, magnesium stearate, medium-chain triglycerides, poloxamer, polyethylene glycol, sodium benzoate, sodium chloride, sodium lauryl sulphate, sodium stearyl fumarate, stearic acid, talc, sucrose stearate, and zinc stearate, and mixtures thereof. A preferred lubricant is magnesium stearate.

Suitable disintegrants of the present disclosure include agar, calcium carbonate, alginic acid, calcium phosphate (tribasic), carboxymethylcellulose calcium, carboxymethylcellulose sodium, colloidal silicon dioxide, croscarmellose sodium, crospovidone, docusate sodium, guar gum, low substituted hydroxypropyl cellulose, magnesium aluminium silicate, methylcellulose, microcrystalline cellulose, sodium alginate, sodium starch glycolate, polacrilin potassium, silicified microcrystalline cellulose, starch and pre-gelatinized starch, and mixtures thereof. The disintegrant may be a combination of disintegrants and/or at least two disintegrants are present, for example a combination of sodium carboxymethyl starch and sodium starch glycolate, such as the sodium starch glycolate sold under the trade name Explotab™. A preferred disintegrant is sodium starch glycolate, in particular Explotab™.

The solid dispersions themselves, and the pharmaceutical compositions and solid dosage forms comprising the solid dispersions of the invention can be used for the treatment of Parkinson's disease in a patient. The solid dispersion can be for use in increasing opicapone bioavailability in a patient suffering from Parkinson's disease, as compared to the opicapone bioavailability which would be obtained from an equivalent medicinal product comprising micronised crystalline opicapone.

The solid dispersions of the invention can be used to manufacture a medicinal product comprising a solid dispersion as defined above, for use in increasing opicapone bioavailability in a patient suffering from Parkinson's disease, as compared to the opicapone bioavailability which would be obtained from an equivalent medicinal product comprising micronised crystalline opicapone.

The solid dispersions of the invention can be used in a method of increasing opicapone bioavailability in a patient suffering from Parkinson's disease comprising administering to said patient a medicinal product comprising a therapeutically effective amount of the solid dispersion, wherein said medicinal product provides increased opicapone bioavailability, as compared to an equivalent medicinal product micronised crystalline opicapone.

### D. Method of Manufacture

Solid dispersions were obtained by the solvent evaporation method, using a Rotary Evaporator Buchi R-300 (5080). Tetrahydrofuran was the solvent used to solubilize both API and excipients for batches Samples 1 to 3, 5 and 10 to 16, which were presented at 0.6% (w/v). For Samples 4, 6 to 9 the solvent used was a mixture of dichloromethane:methanol 50:50 (v:v), and the solids represented 1.5% of the total volume (w/v). The selection of solvent is not particularly limited and only require that the API, polymers, surfactants and other excipients are soluble, and that the solvent can be removed under negative pressure.

Evaporation process conditions of the rotary evaporator used to obtain solid dispersions are described in Table C.

TABLE C
Evaporation process conditions.

| Solvent | Tetrahydrofuran | | dichloromethane:methanol | |
|---|---|---|---|---|
| Parameter | Phase 1 | Phase 2 | Phase 1 | Phase 2 |
| Bath Temperature (° C.) | 40 | 40 | 40 | 40 |
| Rotations (rpm) | 100 | 100 | 80 | 80 |
| Pressure (mbar) | 200 | 0 | 165 | 0 |
| Cooling Temperature (° C.) | 10 | 10 | 10 | 10 |
| Time (h) | 1 | 1 | 1 | 1 |

The solvent was evaporated in stage 1, and the solids were formed in the walls of the evaporation flask. In stage 2, a more efficient drying was achieved.

The solids were collected in a Petri dish, dried at 40° C. in vacuum atmosphere, until constant weight, grinded and sieved (250 μm). Milled material was dried at 40° C. in vacuum atmosphere, until constant weight and evaluated by visual inspection, SEM, XRPD, HPLC and kinetic solubility (FaSSIF and FaSSGF).

### E. Analytical Methods

### Scanning Electron Microscopy (SEM)

A Phenom Pro-X Electron microscope (PhenomWorld, Thermo Fisher Scientific, USA) equipped with a CeB₆ electron source and backscatter electron detector was used to determine the morphology and particle size of the samples.

The powder was placed on conductive adhesive tape. The holder for non-conductive samples was used. The excess of sample (loosely bound to the tape) was removed using compressed air.

Particle morphology was assessed by Automated Image Mapping software and particle size determination was made by Particlemetric software.

### X-Ray Powder Diffraction (XRPD)

Powder X-ray diffraction (PXRD) determinations were performed using a table-top diffractometer MiniFlex 600 (Rigaku, Japan) with a D/teX Ultra detector. In all measurements Cu Kα radiation (40 kV, 15 mA) was used.

The level of remaining crystalline opicapone was assess by comparing the integrated peak intensities of the solid dispersions to those of micronised crystalline opicapone polymorph A with a level less than 30% considered acceptable. Generally, the level of amorphous opicapone was greater than 70%, in most cases over 80% and ideally over 90%. This is equivalent to generally less than 30% crystalline opicapone, in most cases less than 20% crystalline opicapone and ideally less than 10% crystalline opicapone.

### High Performance Liquid Chromatography (HPLC)

The content of opicapone in kinetic solubility studies was analysed by HPLC coupled to a UV/Vis detector (Waters series, USA). A C18 column was used. The mobile phase consisted of 0.1% o-phophoric acid and acetonitrile (68:32 v/v).

For assay and purity determination, a C18 column or a RP8 column were used.

### Stability Testing

Stability testing was performed in double polyethylene (PE) zipper bags, at 70° C. and 75% RH for 19 days. Samples were tested by visual inspection, XRPD and assayed for purity by HPLC.

Furthermore, long-term stability testing was performed at room relative humidity (RH) of 60% RH and temperature (25° C.) for 6 or 12 months in amber glass bottles. Samples were tested by visual inspection, XRPD and assayed for purity by HPLC. Such tests are more relevant for the actual stability of the active pharmaceutical ingredient, opicapone.

### Fasted State Simulated Gastric Fluid (FaSSGF) Kinetic Solubility Assay

Kinetic solubility studies on the opicapone solid dispersion were performed at room temperature at a loading equivalent to˜10 mg of opicapone per mL of FaSSGF, in a total of 5 mL of medium. Samples were tested at 0, 0.5, 1, 2, 4 and 24 hours (Klein, AAPS J., 2010, 12, 397-406).

### Fasted State Simulated Intestinal Fluid (FaSSIF) Kinetic Solubility Assay

Kinetic solubility studies on the opicapone solid dispersion were performed at room temperature at a loading equivalent to˜75 mg of opicapone per mL of FaSSIF, in a total of 5 mL of medium. Samples were tested at 0, 0.5, 1, 2, 4 and 24 hours (Klein, AAPS J., 2010, 12, 397-406).

### Calculation of FaSSGF and FaSSIF Pharmacokinetic Parameters

Area under the Concentration Curve (AUC), Cmax and other pharmacokinetic parameters were calculated using GraphPad Prism 7 for Windows (GraphPad Software, USA).

### In Vivo Pharmacokinetics

The pharmacokinetics of solid dispersions of amorphous opicapone were evaluated in male Wistar rats, after oral administration of Sample 1, 4 or 6, or a control sample of micronised crystalline opicapone, at a dose of 3 mg/kg of opicapone. The exposure of these solid dispersions, formulated as a HPMC suspensions (50 mg of opicapone per 100 mL of 0.2% HPMC), were compared with the exposure of micronised crystalline opicapone, at a dose of 3 mg/kg of opicapone.

During the studies, blood was collected at different time points, from tail vein, spun at 1500×g in a refrigerated centrifuge (4° C.) for 15 min, and the plasma obtained was stored at −80° C. until further analysis. The plasma samples collected from twenty animals (140 samples), were analysed for opicapone exposure. The bioanalysis involved the use of LC-MS/MS after plasma precipitation.

### F. Examples

Opicapone was synthesised as described in WO 2013/089573, which also discloses suitable micronisation methods. Micronised crystalline opicapone was produced using an MC JETMILL®200 with a feed rate between 50 and 300 g/30 s and a milling pressure of 3.6 to 10 bar. with the following particle size distribution (equivalent circular diameter): D10≥4 μm, D50 10-40 μm and D95≤80 μm.

The polymers Plasdone S630 (Ashland), Kollidon 12PF (BASF), HPMCAS (Ashland) and HPMC ES (Dow) were purchased from the commercial suppliers indicated.

The surfactants Kolliphor P188, Kolliphor P407, Kolliphor TPGS, SLS (Texapon K12P) and polysorbate 80 (Tween-80) were purchased from BASF and Gelucire 44/14 was purchased from Gattefossé.

Experiment 1—Production of Solid Dispersions with a Variety of Polymers

The following solid dispersions shown in Table 1 were produced using the method described in section D, above.

TABLE 1
Composition of solid dispersions on amorphous opicapone and
PSD (circle equivalent diameter) by SEM.

| | | Polymer | D10 | D50 | D90 |
|---|---|---|---|---|---|
| Sample | Polymer | class | (μm) | (μm) | (μm) |
| 1 | Plasdone S630 (1:4) | PVP:PVA | 69.9 | 162 | 258 |
| 2 | OPC:Kollidon 12PF (1:4) | PVP | 51.5 | 122 | 216 |
| 3 | OPC:HPMCAS HG (1:4) | HPMCAS | 96.1 | 196 | 265 |
| 4 | OPC:HPMC E5 (1:4) | HPMC | 94.8 | 244 | 344 |

Batches of opicapone amorphous solid dispersions (Samples 1 to 4) were characterised by SEM, XRPD, HPLC, FaSSGF and FaSSIF.

The SEM analysis (FIG. 1 and FIG. 2) confirmed that the solid dispersions were free of large agglomerates and that the particles had equivalent circular diameters disclosed in Table 1.

The XRPD analysis (FIG. 3) confirmed the samples were largely amorphous, due to a general absence of sharp peaks. Sample 2 displayed a low level of crystallinity with small peaks between 5 and 15 deg. It was estimated that crystalline opicapone accounted for less than 30% of the total sample, in most cases less than 10%.

The kinetic solubility over 24 hours was tested in a FaSSGF kinetic solubility assay, which mimics solubility in the stomach. FIG. 4 confirms that solid dispersion of amorphous opicapone in a various polymers gave between 500% and 1,400% kinetic solubility at 4 hours in FaSSGF paradigm compared to a standard control of micronised crystalline opicapone. Therefore, production of amorphous solid dispersion of opicapone generally enhances kinetic solubility under conditions similar to the stomach. This is despite micronised crystalline opicapone having enhanced solubility and bioavailability compared to the non-micronised form. In the FaSSGF kinetic solubility assay HPMC displayed a particularly strong increase compared to other polymers. This might allow solid dispersions comprising HPMC to pass more rapidly to the intestine, where opicapone is primarily absorbed. Therefore, solid dispersions comprising an amount of HPMC are preferred.

Based on the FaSSGF kinetic solubility assay, pharmacokinetic parameters were estimated and are summarised in Table 2, below.

TABLE 2
Pharmacokinetics parameters calculated for kinetic solubility in FaSSGF.

| | Opicapone | Sample 1 | Sample 2 | Sample 3 | Sample 4 |
|---|---|---|---|---|---|
| Parameter | Control | PVP:PVA | PVP | HPMCAS | HPMC |
| tₘₐₓ (h) | 4 | 0.5 | 0.5 | 0.5 | 0.5 |
| Cₘₐₓ (mg/mL) | 0.0003 | 0.0102 | 0.0082 | 0.0040 | 0.0087 |
| AUC (mg * h/mL) | 0.007625 | 0.0483 | 0.04336 | 0.04432 | 0.0672 |
| Cₘₐₓ ratio | N/A | 34× | 27× | 13× | 29× |
| AUC ratio | N/A | 6.3× | 5.7× | 5.8× | 8.8× |

This model confirms that Cₘₐₓ is increased between 13 and 34 times and the AUC increase between 5.7 and 8.8 times in the solid dispersions compared to micronised crystalline opicapone. This suggests the dose of opicapone in formulations comprising solid dispersions could be substantially lower, i.e. between 2 and 30 times lower, preferably, 5 to 20 times lower.

Furthermore, the tₘₐₓ is reduced in all samples, confirming the solid dispersions dissolve in the stomach much quicker. This should ensure opicapone passes to the intestine rapidly for absorption.

Importantly, the AUC ratio was consistently increased by 5 to 9 times compared to micronised crystalline opicapone.

The kinetic solubility over 24 hours was then tested in a FaSSIF kinetic solubility assay, which mimics solubility in the intestine. FIG. 5 confirms that solid dispersion of amorphous opicapone in a various polymers gave between 127% and 1,500% kinetic solubility at 4 hours in FaSSIF paradigm compared to a standard control of micronised crystalline opicapone. The effect was particularly strong for the PVP:PVA and PVP polymers, especially the PVP:PVA copolymer. Therefore, solid dispersions comprising PVP:PVA are particularly preferred because they maximise kinetic solubility at the believed site of opicapone absorption and are predicted to maximise bioavailability.

Based on full kinetic analysis of the FaSSIF data, shown in FIG. 5, pharmacokinetic parameters in FaSSIF were estimated and are summarised in Table 3, below.

TABLE 3
Pharmacokinetics parameters calculated for kinetic solubility in FaSSIF.

| | Opicapone | Sample 1 | Sample 2 | Sample 3 | Sample 4 |
|---|---|---|---|---|---|
| Parameter | Control | PVP:PVA | PVP | HPMCAS | HPMC |
| tₘₐₓ (h) | 24 | 0.5 | 0.5 | 0.5 | 0.5 |
| Cₘₐₓ (mg/mL) | 0.069 | 3.09 | 0.671 | 0.142 | 0.459 |
| AUC | 1.589 | 25.21 | 10.65 | 1.714 | 2.715 |
| (mg * h/mL) | | | | | |
| Cₘₐₓ ratio | N/A | 45× | 9.7× | 2.1× | 6.7× |
| AUC ratio | N/A | 16× | 6.7× | 1.1× | 1.7× |

This model confirms that Cₘₐₓ is increased between 2 and 45 times and the AUC 1.1 to 16 times in the solid dispersions compared to micronised crystalline opicapone. This suggests the dose of opicapone in formulations comprising solid dispersions could be substantially lower, i.e. between 2 and 40 times lower, preferably, 5 to 20 times lower.

Furthermore, the tₘₐₓ is reduced in all samples, confirming the solid dispersions dissolve in the intestine much quicker. This should ensure opicapone bioavailability is maximised and the dose can be minimised with clear advantages in terms of cost and number of patients that can be treated.

Importantly, the AUC ratio was particularly increase for PVP:PVA copolymer compared to other polymers and micronised crystalline opicapone.

To confirm the FaSSGF and FaSSIF pharmacokinetic parameters were relevant to the in vivo situation, the pharmacokinetic behaviour of three representative samples (Sample 1, Sample 4 and Sample 6 (discussed in Experiment 2, below)) were tested in male Wistar rats. In agreement with the in vitro models, all samples displayed improved pharmacokinetic parameters compared to micronised crystalline opicapone (FIG. 6 and Table 4).

TABLE 4
Pharmacokinetics parameters calculated in rat model.

| Parameter | Opicapone | | Sample 1 | | Sample 6 | | Sample 4 | |
|---|---|---|---|---|---|---|---|---|
| tₘₐₓ (h) | 2 | (1-4) | 0.5 | | 0.5 | (0.5-4) | 1 | (0.5-1) |
| t_{1/2} (h) | * | | 1.3 | (32.1) | 0.9 | (22.5)** | 1.1 | (23.3) |
| Cₘₐₓ (ng/mL) | 379.3 | (68.9) | 3808.6 | (29.9) | 3918.5 | (92.7) | 1395.6 | (83.6) |
| tₗₐₛₜ (h) | 8 | (4-8) | 4 | | 4 | (4-8) | 8 | (4-8) |
| Cₗₐₛₜ (ng/mL) | 78.6 | (111.9) | 242.0 | (65.4) | 84.6 | (93.9) | 36.4 | (86.8) |
| AUCₗₐₛₜ (mg * h/mL) | 1400.2 | (70.4) | 3926.0 | (23.8) | 4628.2 | (65.2) | 3811.4 | (29.9) |

| | | | | | | | |
|---|---|---|---|---|---|---|---|
| AUC_{0-inf} | * | 4439.5 | (27.2) | 6200.8 | (74.5) | 3862.3 | (29.4) |

| | | | | |
|---|---|---|---|---|
| Cₘₐₓ ratio | NA | 10.0 | 10.3 | 3.7 |
| AUCₗₐₛₜ ratio | NA | 2.8 | 3.3 | 2.7 |

*Not calculated (not enough values in terminal phase to calculate the slope
**3 out of 5 values used

As predicted, the PVP:PVA copolymer (Sample 1), with improved absorbance in the intestine displayed a greater enhancement compared to HPMC (Sample 4). However, the HPMC polymer still showed significant increases in bioavailability compared to micronised crystalline opicapone, demonstrating the importance of good kinetic solubility in the stomach. These results confirm that amorphous solid dispersions of opicapone have improved kinetic solubility and bioavailability compared to micronised crystalline opicapone.

As discussed in Experiment 2, below, the amorphous solid dispersions of opicapone displayed good chemical stability.

Experiment 2—Production of Solid Dispersions with Mixtures of PVP:PVA and HPMC Polymers

Based on the discovery that (a) solid dispersions of amorphous opicapone had improved kinetic solubility and pharmacokinetic parameters compared to a standard control of micronised crystalline opicapone, and (b) that the level of improvement varied, the inventors performed further experiments to identify combinations of polymers that resulted in particularly large increases in kinetic solubility and bioavailability. The inventors prepared solid dispersions comprising amorphous opicapone and different polymer mixtures.

The following solid dispersions shown in Table 5 were produced using the method described in section D, above.

TABLE 5
Composition of solid dispersions on amorphous opicapone
and PSD (circle equivalent diameter) by SEM.

| | Opicapone/ | Polymer | | | |
|---|---|---|---|---|---|
| | PVP:PVA/ | PVP:PVP | D10 | D50 | D90 |
| Sample | HPMC ratio | (%) | (μm) | (μm) | (μm) |
| 5 | 1/4/0 | 100 | 69.9 | 155 | 303 |
| 6 | 1/3/1 | 75 | 42.3 | 105 | 197 |
| 7 | 1/2/2 | 50 | 72.6 | 149 | 240 |
| 8 | 1/1/3 | 25 | 87.9 | 187 | 307 |
| 9 | 1/0/4 | 0 | 110 | 241 | 346 |

Batches of opicapone amorphous solid dispersions (Samples 5 to 9) were characterised by SEM, XRPD, HPLC, FaSSGF and FaSSIF.

The SEM analysis (FIG. 7 and FIG. 8) confirmed that the solid dispersions were free of large agglomerates and that the particles had equivalent circular diameters disclosed in Table 5.

The XRPD analysis (FIG. 9) confirmed the samples were largely amorphous, due to a general absence of sharp peaks. The samples displayed a low level of crystallinity with small peaks between 5 and 15 deg. It was estimated that crystalline opicapone accounted for less than 30% of the total sample, in most cases less than 10%.

The kinetic solubility over 24 hours was tested in a FaSSGF kinetic solubility assay, which mimics solubility in the stomach. FIG. 10 confirms that solid dispersion of amorphous opicapone with mixtures of PVP:PVA and HPMC gave between 700% and 2,100% kinetic solubility at 4 hours in FaSSGF paradigm compared to a standard control of micronised crystalline opicapone. Surprisingly, production of amorphous solid dispersion of opicapone comprising a mixture of PVP:PVA and HPMC enhances kinetic solubility under conditions similar to the stomach greater than either polymer alone (especially compared to PVP:PVA alone) and micronised crystalline opicapone. Therefore, this combination results in a synergistic enhancement in kinetic solubility. This might allow solid dispersions comprising PVP:PVA and HPMC to pass more rapidly to the intestine, where opicapone is primarily absorbed. Therefore, solid dispersions comprising an amount of HPMC are preferred.

Based on the FaSSGF kinetic solubility assay, pharmacokinetic parameters were estimated and are summarised in Table 6, below.

TABLE 6
Pharmacokinetics parameters calculated
for kinetic solubility in FaSSGF.

| | | Sample | Sample | Sample | Sample | Sample |
|---|---|---|---|---|---|---|
| Parameter | Opicapone | 5 | 6 | 7 | 8 | 9 |
| tₘₐₓ (h) | 24 | 1 | 0.5 | 0.5 | 0.5 | 0.5 |
| Cₘₐₓ | 0.0003 | 0.0084 | 0.0067 | 0.0082 | 0.0069 | 0.0083 |
| (mg/mL) | | | | | | |
| AUC | 0.00575 | 0.0399 | 0.06815 | 0.07178 | 0.07568 | 0.05193 |
| (mg*h/mL) | | | | | | |
| Cₘₐₓ ratio | N/A | 28x | 22x | 27x | 23x | 28x |
| AUC ratio | N/A | 7x | 12x | 12x | 13x | 9x |

This model confirms that Cₘₐₓ is increased between 22 and 28 times and the AUC increase between 7 and 13 times in the solid dispersions compared to micronised crystalline opicapone. This suggests the dose of opicapone in formulations comprising solid dispersions could be substantially lower, i.e. between 2 and 30 times lower, preferably, 5 to 20 times lower.

Furthermore, the tₘₐₓ is reduced in all samples, confirming the solid dispersions dissolve in the stomach much quicker. This should ensure opicapone passes to the intestine rapidly for absorption.

Importantly, the AUC ratio was consistently increased by 7 to 13 times compared to micronised crystalline opicapone, especially in samples comprising mixtures of PVP:PVA and HPMC.

The kinetic solubility over 24 hours was also tested in a FaSSIF kinetic solubility assay, which mimics solubility in the intestine. FIG. 11 confirms that solid dispersion of amorphous opicapone with mixtures of PVP:PVA and HPMC gave between 1,100% and 2,300% kinetic solubility at 4 hours in FaSSIF paradigm compared to a standard control of micronised crystalline opicapone. Surprisingly, production of amorphous solid dispersion of opicapone comprising a mixture of PVP:PVA and HPMC enhances kinetic solubility under conditions similar to the intestine greater than either polymer when more than 25% PVP:PVA was included (i.e. a 1:3 ratio of PVP:PVA to HPMC or a 1/1/3 combination of opicapone/PVP:PVA/HPMC). Therefore, this combination results in a synergistic enhancement in kinetic solubility in the intestine. This might allow solid dispersions comprising PVP:PVA and HPMC to dissolve more rapidly to the intestine, where opicapone is primarily absorbed. In the FaSSIF kinetic solubility assay a 1/3/1 combination of opicapone/PVP:PVA/HPMC (i.e. a 3:1 ratio of PVP:PVA to HPMC or 75% PVP:PVA) also displayed a particularly strong increase compared to other polymers. Therefore, solid dispersions comprising this ratio of PVP:PVA to HPMC are particularly preferred when maximum kinetic solubility in the intestine is required.

The inventors discovered that when the kinetic solubility at 4 hours in the FaSSIF kinetic solubility assay was plotted against the amount of PVP:PVA to HPMC (in percentage PVP:PVA), a clear parabolic trend emerged with an apex around the 3:1 PVP:PVA to HPMC ratio (i.e. 75% PVP:PVA). This is shown in FIG. 12. This trend can be used to predict the optimal levels of bioavailability based on the relative amounts of these polymers in the solid dispersions of amorphous opicapone. Generally, the aim is to maximise bioavailability, but the relationship could be used to modulate bioavailability of opicapone solid dispersions to ensure bioequivalence with a marketed formulation, for example, micronised crystalline opicapone.

Based on the FaSSIF kinetic solubility assay, pharmacokinetic parameters were estimated and are summarised in Table 7, below.

TABLE 7
Pharmacokinetics parameters calculated
for kinetic solubility in FaSSIF.

| | | Sample | Sample | Sample | Sample | Sample |
|---|---|---|---|---|---|---|
| Parameter | Opicapone | 5 | 6 | 7 | 8 | 9 |
| tₘₐₓ (h) | 2 | 0.5 | 0.5 | 1 | 0.5 | 1 |
| Cₘₐₓ | 0.086 | 8.462 | 2.775 | 2.562 | 2.033 | 0.751 |
| (mg/mL) | | | | | | |
| AUC | 1.31 | 31.9 | 46.9 | 34.2 | 18.1 | 3.95 |
| (mg*h/mL) | | | | | | |
| Cₘₐₓ ratio | N/A | 98x | 32x | 30x | 24x | 8.7x |
| AUC ratio | N/A | 24x | 36x | 26x | 14x | 3x |

This model confirms that Cₘₐₓ is increased between 8.7 and 98 times and the AUC increased 3 to 36 times in the solid dispersions including a mixture of PVP:PVA and HPMC compared to micronised crystalline opicapone. This suggests the dose of opicapone in formulations comprising these polymer mixtures could be substantially lower, i.e. between 2 and 40 times lower, preferably, 5 to 20 times lower.

Furthermore, the tₘₐₓ is reduced in all samples, confirming the solid dispersions dissolve in the intestine much quicker. This should ensure opicapone bioavailability is maximised and the dose can be minimised with clear advantages in terms of cost and number of patients that can be treated.

Importantly, the AUC ratio was particularly increased for the mixtures of PVP:PVA and HPMC compared to HPMC and micronised crystalline opicapone.

To confirm the FaSSGF and FaSSIF pharmacokinetic parameters were relevant to the in vivo situation, the pharmacokinetic behaviour of Sample 6 was tested in male Wistar rats. In agreement with the in vitro models, this Sample 6 displayed improved pharmacokinetic parameters compared to micronised crystalline opicapone (FIG. 6 and Table 4).

To confirm the samples were stable in the presence of PVP:PVA and/or HPMC, provisional experiments were performed on Samples 5, 6 and 9 at 70° C. and 75% RH for 19 days. This confirmed that all samples maintained good chemical stability with total impurities increasing from 0.1-0.5% at day 0 to 3.4-4.3% by day 19. This confirmed that the opicapone remained 95.7-96.6% pure and that all opicapone amorphous solid dispersions remained chemically stable in spite of their different kinetic solubility.

Furthermore, long-term stability testing was performed at room relative humidity (RH) of 60% RH and temperature (25° C.) for 12 months in amber glass bottles. Samples 5, 6 and 9 were tested by visual inspection, XRPD and assayed for purity by HPLC. Such tests are more relevant for the actual stability of the active pharmaceutical ingredient, opicapone. The results are shown in Table 8, below.

TABLE 8
long-term stability of samples at room relative humidity (RH) of 60%
RH and temperature (25° C.) for 12 months in amber glass bottles.

| Parameter | Sample 5 | Sample 6 | Sample 9 |
|---|---|---|---|
| Description | Yellow solid | Yellow solid | Yellow solid |
| Crystallinity (XRPD) | Amorphous | ~20% crystalline | ~20% crystalline |
| Total impurity (%) | 0.4 | 0.1 | 0.5 |

This confirmed only 0.1 to 0.5% opicapone-related impurities were present and that the opicapone remained 99.5 to 99.9% pure. Whilst all samples displayed excellent chemical stability and retained their yellow colour, only Sample 5 (PVP:PVA) fulfilled the strictest definition of “amorphous opicapone”, defined above, whereas other samples showed signs of crystalline content (˜20%). Sample 5 displayed no detectable peaks related to opicapone in the XRPD spectrum between 2 and 40 degrees after long-term storage under these conditions. Therefore, samples in which PVP:PVA is the primary polymer are expected to maintain good bioavailability over longer periods compared to the more kinetically soluble samples mixtures of PVP:PVA and HPMC.

Experiment 3—Production of Solid Dispersions with Surfactants

The following solid dispersions shown in Table 9 were produced using the method described in section D, above.

TABLE 9
PSD (circle equivalent diameter) by SEM for solid dispersions
of OPC including different surfactants.

| | | D10 | D50 | D90 |
|---|---|---|---|---|
| Sample | Surfactant | (μm) | (μm) | (μm) |
| 10 | Opicapone:Plasdone S630 | 83.6 | 185 | 328 |
| | (1:4) | | | |
| 11 | Opicapone:Plasdone S630:Gelucire 44/14 | 55.6 | 141 | 252 |
| | (1:3.75:0.25) | | | |
| 12 | Opicapone:Plasdone S630:Kolliphor P188 | 65.0 | 153 | 258 |
| | (1:3.75:0.25) | | | |
| 13 | Opicapone:Plasdone S630:Kolliphor P407 | 76.1 | 171 | 308 |
| | (1:3.75:0.25) | | | |
| 14 | Opicapone:Plasdone S630:Kolliphor TPGS | 74.4 | 162 | 267 |
| | (1:3.75:0.25) | | | |
| 15 | Opicapone:Plasdone S630:SLS | 66.6 | 154 | 240 |
| | (1:3.75:0.25) | | | |
| 16 | Opicapone:Plasdone S630:Tween 80 | 39.8 | 94.7 | 186 |
| | (1:3.75:0.25) | | | |

Batches of opicapone amorphous solid dispersions (Samples 10 to 16) were characterised by SEM, XRPD, HPLC, FaSSGF and FaSSIF.

The SEM analysis (FIG. 13 and FIG. 14) confirmed that the solid dispersions were free of large agglomerates and that the particles had equivalent circular diameters disclosed in Table 9.

The XRPD analysis (FIG. 15) confirmed the samples were largely amorphous, due to a general absence of sharp peaks. Sample 15 (SLS surfactant) displayed a low level of crystallinity with small peaks between 30 and 40 deg. It was estimated that crystalline opicapone accounted for less than 30% of the total sample and in most cases less than 10%.

The kinetic solubility over 24 hours was tested in a FaSSGF kinetic solubility assay, which mimics solubility in the stomach. FIG. 16 confirms that solid dispersion of amorphous opicapone with mixtures with a variety of surfactants gave excellent kinetic solubility. Solid dispersion of amorphous opicapone with mixtures of surfactants displayed between 200% and 1,600% kinetic solubility at 4 hours in FaSSIF paradigm compared to a standard control of micronised crystalline opicapone. Surprisingly, Sample 13 (containing vitamin E polyethylene glycol succinate) showed particular enhancement compared to the surfactant-free solid dispersion. Therefore, this class of surfactants further enhance kinetic solubility in the stomach. This might allow solid dispersions comprising vitamin E polyethylene glycol succinate to pass more rapidly to the intestine, where opicapone is primarily absorbed. Therefore, solid dispersions comprising an amount of vitamin E polyethylene glycol succinate are preferred.

Based on the FaSSGF kinetic solubility assay, pharmacokinetic parameters were estimated and are summarised in Table 10, below.

TABLE 10
Pharmacokinetics parameters calculated for kinetic solubility in FaSSGF.

| | | Sample | Sample | Sample | Sample | Sample | Sample | Sample |
|---|---|---|---|---|---|---|---|---|
| Parameter | opicapone | 10 | 11 | 12 | 13 | 14 | 15 | 16 |
| tₘₐₓ (h) | 24 | 0.5 | 0.5 | 0.5 | 0.5 | 0.5 | 0.5 | 0.5 |
| Cₘₐₓ | 0.00026 | 0.0082 | 0.0035 | 0.0085 | 0.0082 | 0.0234 | 0.0072 | 0.0018 |
| (mg/mL) | | | | | | | | |
| AUC | 0.00586 | 0.03574 | 0.0275 | 0.03366 | 0.03795 | 0.1244 | 0.02042 | 0.01083 |
| (mg*h/mL) | | | | | | | | |
| Cₘₐₓ ratio | N/A | 32x | 13x | 33x | 32x | 90x | 28x | 6.9x |
| AUC ratio | N/A | 6.1x | 4.7x | 5.7x | 6.5x | 21x | 3.5x | 1.8x |

This model confirms that Cₘₐₓ is increased between 6.9 and 90 times and the AUC increased 1.8 to 21 times in the solid dispersions including a mixture of PVP:PVA and HPMC compared to micronised crystalline opicapone. This suggests the dose of opicapone in formulations comprising these polymer mixtures could be substantially lower, i.e. between 2 and 40 times lower, preferably, 5 to 20 times lower.

Furthermore, the tₘₐₓ is reduced, confirming the solid dispersions dissolve in the stomach much quicker. This should ensure opicapone passes to the intestine rapidly for absorption.

The kinetic solubility over 24 hours was tested in a FaSSIF kinetic solubility assay, which mimics solubility in the intestine. FIG. 17 confirms that solid dispersion of amorphous opicapone with mixtures with a variety of surfactants gave excellent kinetic solubility with the exception of Sample 15 (SLS). Excluding this embodiment, solid dispersion of amorphous opicapone with mixtures of surfactants displayed between 1,200% and 1,800% kinetic solubility at 4 hours in FaSSIF paradigm compared to a standard control of micronised crystalline opicapone. Surprisingly, Sample 11 (containing the lauroyl macroglyceride Gelucire 44/14), Sample 12 (containing the poloxamer Poloxamer 188) and in particular Sample 16 (containing the polysorbate, Polysorbate 80) showed particular enhancement compared to the surfactant-free solid dispersion. Therefore, these classes of surfactants further enhance kinetic solubility in the intestine. This might allow solid dispersions comprising these surfactants to dissolve more rapidly to the intestine, where opicapone is primarily absorbed. Therefore, solid dispersions comprising these surfactants, especially polysorbates such as polysorbate 80 are particularly preferred.

It is clear from the results above that SLS is less compatible with solid dispersions of amorphous opicapone. However, a broad range of non-ionic surfactants from different classes are compatible with the solid dispersions of the invention and even enhance the kinetic solubility of opicapone. Less active embodiments can be avoided by excluding anionic surfactants, especially organosulphate surfactant, specifically, SLS.

Based on the FaSSIF kinetic solubility assay, pharmacokinetic parameters were estimated and are summarised in Table 11, below.

TABLE 11
Pharmacokinetics parameters calculated for kinetic solubility in FaSSIF.

| | | Sample | Sample | Sample | Sample | Sample | Sample | Sample |
|---|---|---|---|---|---|---|---|---|
| Parameter | opicapone | 10 | 11 | 12 | 13 | 14 | 15 | 16 |
| tₘₐₓ (h) | 4 | 1 | 0.5 | 1 | 1 | 1 | 4 | 1 |
| Cₘₐₓ | 0.089 | 1.656 | 1.836 | 1.833 | 1.794 | 1.638 | 0.036 | 2.005 |
| (mg/mL) | | | | | | | | |
| AUC | 1.40 | 31.5 | 35.6 | 36.7 | 30.8 | 29.5 | 0.60 | 38.3 |
| (mg*h/mL) | | | | | | | | |
| Cₘₐₓ ratio | N/A | 19x | 21x | 21x | 20x | 18x | 0.4x | 23x |
| AUC ratio | N/A | 23x | 25x | 26x | 22x | 21x | 0.4x | 27x |

This model confirms that Cₘₐₓ is increased between 18 and 23 times and AUC increased 22 to 27 times in the solid dispersions compared to micronised crystalline opicapone, when SLS is excluded. This suggests the dose of opicapone in formulations comprising solid dispersions could be substantially lower, i.e. between 2 and 30 times lower, preferably, 5 to 20 times lower.

Furthermore, the tₘₐₓ is reduced, when SLS is excluded, confirming the solid dispersions dissolve in the intestine much quicker.

Importantly, the AUC ratio was consistently increased by 18 to 23 times compared to micronised crystalline opicapone, when SLS is excluded.

To confirm the samples were stable in the presence of different surfactants, provisional experiments were performed on Samples 10 to 14 at 70° C. and 75% RH for 19 days. This confirmed that all samples maintained good chemical stability with total impurities increasing from 0.3-0.4% at day 0 to 2.9-5.1% by day 19. This confirmed the opicapone remained 94.9-97.1% pure and that all opicapone amorphous solid dispersions remained chemically stable in spite of their different kinetic solubility.

Furthermore, long-term stability testing was performed at room relative humidity (RH) of 60% RH and temperature (25° C.) for 6 months in amber glass bottles. Samples 10 to 14 were tested by visual inspection, XRPD and assayed for purity by HPLC. Such tests are more relevant for the actual stability of the active pharmaceutical ingredient, opicapone.

TABLE 12
long-term stability of samples at room relative humidity (RH) of 60%
RH and temperature (25° C.) for 6 months in amber glass bottles.

| Parameter | Sample 10 | Sample 11 | Sample 12 | Sample 13 | Sample 14 |
|---|---|---|---|---|---|
| Description | Yellow | Yellow | Yellow | Yellow | Yellow |
| | solid | solid | solid | solid | solid |
| Crystallinity | Amor- | Amor- | Amor- | Amor- | Amor- |
| (XRPD) | phous | phous | phous | phous | phous |
| Total | 0.4 | 0.4 | 0.4 | 0.4 | 0.3 |
| impurity | | | | | |
| (%) | | | | | |

This confirmed only 0.3 to 0.4% opicapone-related impurities were present and that the opicapone remained 99.6 to 99.7% pure. All samples displayed excellent chemical stability, retained their yellow colour and fulfilled the strictest definition of “amorphous opicapone”, defined above. Samples 10 to 14 displayed no detectable peaks related to opicapone in the XRPD spectrum between 2 and 40 degrees after long-term storage under these conditions. In particular, the results were highly similar to PVP:PVA without surfactant. Therefore, the presence of surfactants enhanced kinetic solubility, but did not reduce the chemical or solid state stability of opicapone in the amorphous solid dispersions.

Based on the results above, it is believed that the surfactants used in Experiment 3 and the PVP:PVA and HPMC mixtures used in Experiment 2 would be mutually compatible and could provide even greater benefits in combination in terms of kinetic stability and bioavailability. Likewise, it is believed that the surfactants used in Experiment 3 will not reduce the chemical or solid state stability of the solid dispersions described in Experiment 1 and Experiment 2.

Other variations to the disclosed embodiments can be understood and effected by those skilled in the art in practicing the claimed invention, from a study of the drawings, the disclosure, and the appended claims. In the claims, the word “comprising” does not exclude other elements or steps, and the indefinite article “a” or “an” does not exclude a plurality. The mere fact that certain measures are recited in mutually different dependent claims does not indicate that a combination of these measures cannot be used to advantage. Any reference signs in the claims should not be construed as limiting the scope.

1. A solid dispersion comprising amorphous opicapone and one or more polymers, wherein the weight ratio of the amorphous opicapone to the one or more polymers ranges from 1:1 to 1:5.

2. A solid dispersion according to claim 1, wherein the weight ratio of the amorphous opicapone to the one or more polymers ranges from 1:3 to 1:5.

3. A solid dispersion according to claim 1 or claim 2, wherein the weight ratio of the amorphous opicapone to the one or more polymers is about 1:4.

4. A solid dispersion according to any one of claims 1 to 3, wherein the one or more polymers are selected from the group consisting of: a copolymer of N-vinyl-2-pyrrolidone and vinyl acetate (PVP:PVA), hydroxypropylmethylcellulose (HPMC), hydroxypropylmethylcellulose acetyl succinate (HPMCAS) and polyvinylpyrrolidone (PVP).

5. A solid dispersion according to claim 4, wherein the one or more polymers are selected from the group consisting of: PVP:PVA and HPMC.

6. A solid dispersion according to claim 5, wherein the one or more polymers is PVP:PVA.

7. A solid dispersion according to claim 5, wherein the one or more polymers are a mixture of PVP:PVA and HPMC.

8. A solid dispersion according to claim 7, wherein the weight ratio of PVP:PVA to HPMC ranges from 1:4 to 20:1.

9. A solid dispersion according to claim 7 or claim 8, wherein the weight ratio of PVP:PVA to HPMC ranges from 1:1 to 10:1.

10. A solid dispersion according to any one of claims 7 to 9, wherein the weight ratio of PVP:PVA to HPMC ranges from 2:1 to 4:1.

11. A solid dispersion according to any one of claims 7 to 10, wherein the weight ratio of PVP:PVA to HPMC is about 3:1.

12. A solid dispersion according to any preceding claim further comprising a surfactant.

13. A solid dispersion according to claim 12 wherein the amount of surfactant ranges from 2 to 8 wt % based on the total weight of the solid dispersion.

14. A solid dispersion according to claim 12 or claim 13 wherein the surfactant is selected from the group consisting of lauroyl macrogolglycerides, poloxamers, polysorbates, and vitamin E polyethylene glycol succinate.

15. A solid dispersion according to any preceding claim wherein the Cₘₐₓ and/or AUC_{0-24 h} of opicapone is/are increased by at least 25% in fasted state simulated intestinal fluid (FaSSIF) compared to micronized crystalline opicapone.

16. A solid dispersion according to any preceding claim wherein the Cₘₐₓ and/or AUC_{0-24 h} of opicapone is/are increased by at least 25% in fasted state simulated gastric fluid (FaSSGF) compared to micronized crystalline opicapone.

17. A solid dispersion according to any preceding claim wherein the Cₘₐₓ and/or AUC_{0-24 h} of opicapone is/are increased by at least 25% in vivo.

18. A solid dispersion according to any preceding claim wherein the opicapone is chemically stable for 6 months, preferably 12 months, at 25° C. and 60% Relative Humidity.

19. A solid dispersion according to claim 18 wherein the amorphous opicapone remains amorphous for 6 months, preferably 12 months, at 25° C. and 60% Relative Humidity.

20. A pharmaceutical composition comprising a solid dispersion as claimed in any one of claims 1 to 19 and a pharmaceutically acceptable excipient.

21. A solid dosage form comprising a pharmaceutical composition according to claim 20.

22. A solid dosage form according to claim 21 which is a tablet or capsule.

23. A method of modulating opicapone bioavailability by modifying the amounts of PVP:PVA and HPMC polymers in a solid dispersion comprising amorphous opicapone.
